(19) United States
(12) Patent Application Publication
(10) Pub. No.: US 2024/0263254 A1
(43) Pub. Date: Aug. 8, 2024

(54) DEVELOPMENT AND VALIDATION OF A 2-GENE HOST-VIRAL TRANSCRIPTOMIC CLASSIFIER FOR ENHANCED COVID-19 DIAGNOSIS

(71) Applicants: CZ Biohub SF, LLC, San Francisco, CA (US); The Regents of the University of California, Oakland, CA (US)

(72) Inventors: Charles R. Langelier, Pacifica, CA (US); Eran Mick, San Fransisco, CA (US); Jack Albright, San Francisco, CA (US); Angela Oliveira Pisco, San Francisco, CA (US); John A. Kamm, San Francisco, CA (US)

(21) Appl. No.: 18/569,144

(22) PCT Filed: Jul. 1, 2022
(86) PCT No.: PCT/US2022/035981

§ 371 Date: Dec. 11, 2023

Related U.S. Application Data
(60) Provisional application No. 63/218,870, filed on Jul. 6, 2021.

(51) Int. Cl. C12Q 1/70; C12Q 1/6809

(52) U.S. Cl. CPC C12Q 1/70; C12Q 1/6809; C12Q 2600/112; C12Q 2600/158

(57) ABSTRACT

Provided herein are lists of combined host-viral gene markers that can be used for identifying COVID-19 in a subject and/or determining severity of disease. The host-viral diagnostic methods, compositions and systems disclosed herein are used to classify human subjects pre-diagnosis, with or without symptoms of COVID-19, based on the expression levels of the identified gene markers.

### CROSS-REFERENCES TO RELATED APPLICATIONS

The present application claims priority from and is a PCT application of U.S. Provisional Application No. 63/218,870, entitled “Development and Validation Of A 2-Gene Host-Viral Transcriptomic Classifier For Enhanced Covid-19 Diagnosis” filed Jul. 6, 2021, the entire contents of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant Number K23HL138461-01A1, awarded by the National Institutes of Health. The government has certain rights in the invention.

### BACKGROUND

The SARS-COV-2 pandemic has resulted in an unprecedented burden of disease and economic disruption, with the United States amongst the most significantly impacted countries. With over 400,000 deaths and an estimated economic cost of $US 16 trillion dollars in the next decade alone, an improved approach to COVID-19 testing, screening and prognosis is urgently needed. Existing polymerase chain reaction (PCR), antigen and antibody-based diagnostics suffer from challenges with test sensitivity, susceptibility to false positive results from laboratory environmental contamination or cross reactivity.

### BRIEF SUMMARY

We performed upper respiratory transcriptional profiling on 970 patients across three cohorts of patients with acute respiratory illnesses to develop and validate an accurate integrated 2-gene host-based COVID-19 classifier. We tested performance of this classifier alone or in conjunction with detection of SARS-CoV-2 by metagenomic next generation sequencing (mNGS) and then optimized the classifier to perform in conjunction with viral detection in a PCR assay adaptable to existing COVID-19 diagnostic platforms.

Surprisingly, we have discovered that an expression assay for just two or three genetic targets can be used to distinguish between samples from subjects currently infected with SARS-COV-2 (SARS-COV-2+ samples) and samples from subjects who were never infected with SARS-COV-2 or who have cleared a SARS-COV-2 infection (SARS-COV-2− samples) with high accuracy, differentiate COVID-19 disease from non-viral acute respiratory illnesses in patients with an respiratory illness, and allow for evaluation of disease pathophysiology and prediction of clinical outcomes of patients with COVID-19.

In one aspect, a method or assay for determining SARS-COV-2 infection in a human subject is provided. The method has advantages over currently used methods, including fewer false positive and false negative results. In a second aspect, a method of assay for diagnosing COVID-19 disease in a human subject is provided.

In one approach, we describe an assay in which expression of two host genes is measured and is sufficient for evaluation of infection or disease state. In another approach, the expression of two host genes is measured and expression of a SARS-COV-2 gene is also measured.

Provided herein is a method of determining SARS-COV-2 infection in a human subject. The method comprises (a) receiving a biological sample collected from the subject, the biological sample including human RNA from cells of the subject and SARS-COV-2 viral RNA, if present, (b) measuring, in the biological sample, (i) a first gene expression level of a first human gene selected from the group consisting of genes shown in Table 12A-C or Table 13, (ii) a second gene expression level of a second human gene selected from the group consisting of genes shown in Table 12 or Table 13, wherein the second human gene is different from the first human gene, (c) detecting differences, if any, in the in the first gene expression level and the second gene expression level relative to references expression levels characteristic of a human subject who is not infected with SARS-COV-2 and does not have evidence of COVID-19, (d) determining whether the subject is infected with SARS-COV-2 based on the differences, if any, determined in (c). In some approaches, the method further comprises detecting the presence or quantity of SARS-COV-2 viral gene in the biological sample, and determining whether the subject is infected with SARS-COV-2 based on the detection of the SARS-COV-2 viral gene and the differences, if any, determined in step (c).

Also provided herein is a method of diagnosing COVID-19 in a human subject, the method comprising (a) receiving a biological sample collected from the subject, the biological sample including human RNA from cells of the subject and SARS-COV-2 viral RNA, if present, (b) measuring, in the biological sample, (i) a first gene expression level of a first human gene selected from the group consisting of genes shown in Table 12 or Table 13, (ii) a second gene expression level of a second human gene selected from the group consisting of genes shown in Table 12 or Table 13, wherein the second human gene is different from the first human gene, (c) detecting differences in the expression levels of the first and second genes relative to reference expression levels characteristic of a human subject who is not infected with SARS-COV-2 and does not have signs or symptoms of COVID-19 disease, (d) determining whether the subject has COVID-19 disease based on the differences, if any, determined in (c). In some approaches, the method further comprises detecting the presence or quantity of SARS-COV-2 viral gene in the biological sample, and determining whether the subject has COVID-19 based on the detection of the SARS-COV-2 viral gene and the differences, if any, determined in step (c).

In some aspects, the SARS-COV-2 viral gene is selected from the group consisting of an SARS-COV-2 Envelope (E) gene, a SARS-COV-2 Nucleocapsid (N) gene, a Spike (S) gene, an SARS-COV-2 open reading frame 1ab (Orf1ab) gene, or an SARS-COV-2 RNA dependent RNA polymerase (RdRP) gene.

In some approaches, the expression level of the first and second human genes in the biological sample is determined by normalizing a measured expression level with an expression level of a human normalization gene (e.g., RNaseP gene).

In some aspects, the biological sample is a sample comprising cells from the nose, mouth, throat or lower respiratory tract of the subject. In some aspects, the biological sample comprises cells collected from the subject's nose and/or mouth and/or throat and/or lower respiratory tract. In some aspects, the sample is collected using a buccal swab, nasal swab, nasopharyngeal swab, nasopharyngeal wash or aspirate, mid-turbinate swab, oropharyngeal swab, or saliva specimen. In some embodiments, the biological sample comprises fluid from the lungs, such as a broncho-alveolar lavage, or an endotracheal aspirate.

In some aspects, the first human gene is selected from genes listed in Table 12 or Table 13. In some aspects, the first human gene is any one of IFI6, IFI44L, and IFI27 and the second human gene is any one of GSTA2, GBP5, and CCL3. In some aspects, the first human gene is from the left column and the second human gene is the corresponding gene in the right column of Table 11.

In another aspect, provided is a method of diagnosing COVID-19 in a subject, the method comprising (a) receiving a biological sample obtained from the subject, the biological sample including RNA of the subject and potentially RNA of SARS-COV-2, (b) measuring, in the biological sample, a first gene expression level of a first human gene selected from the group consisting of genes shown in Table 12 or Table 13, (c) measuring, in the biological sample, a viral expression level of a SARS-COV-2 viral gene, and (d) determining a classification of whether the subject has COVID-19 using the first gene expression level, the viral expression level, and one or more reference gene expression levels determined from a plurality of reference samples having a known COVID-19 classification. In some aspects, the method further comprises measuring, in the biological sample, a second gene expression level of a second human gene selected from the group consisting of genes shown in Table 12 or Table 13, and determining a classification of whether the subject has COVID-19 using the first gene expression level, the second gene expression level, the viral expression level, and one or more reference gene expression levels determined from a plurality of reference samples having a known COVID-19 classification.

In yet another aspect, provided is a method of diagnosing COVID-19 in a subject, the method comprising (a) receiving a biological sample obtained from the subject, the biological sample including RNA of the subject, (b) measuring, in the biological sample, a first gene expression level of a first human gene selected from the group consisting of genes shown in Table 12 or Table 13, (c) determining a classification of whether the subject has COVID-19 using the first gene expression level and one or more reference gene expression levels determined from a plurality of reference samples having a known COVID-19 classification. Variations of the method may further include measuring, in the biological sample, a second gene expression level of a second human gene selected from the group consisting of genes shown in Table 12 or Table 13, and determining a classification of whether the subject has COVID-19 using the first gene expression level, the second gene expression level, and one or more reference gene expression levels determined from a plurality of reference samples having a known COVID-19 classification.

Provided herein is also a method (e.g., a prognostic method) for determining a prognosis or assessing likely disease outcome in a subject having COVID-19 based on the gene expression levels of gene markers provided herein. In one example, the method comprises (a) receiving a biological sample obtained from the subject, the biological sample including RNA of the subject and potentially RNA of SARS-COV-2, (b) measuring, in the biological sample, a first gene expression level of a first human gene selected from the group consisting of genes shown in Table 12 or Table 13, (c) measuring, in the biological sample, a viral expression level of a SARS-COV-2 viral gene, and (d) determining a classification of COVID-19 disease outcome using the first gene expression level, the viral expression level, and one or more reference gene expression levels determined from a plurality of reference samples having a known COVID-19 disease outcome classification. In some aspects, the method further comprises measuring, in the biological sample, a second gene expression level of a second human gene selected from the group consisting of genes shown in Table 12 or Table 13, and determining a classification of COVID-19 disease outcome using the first gene expression level, the second gene expression level, the viral expression level, and one or more reference gene expression levels determined from a plurality of reference samples having a known COVID-19 disease phenotype outcome (e.g., mild versus severe illness).

In another example, provided is a method for determining a prognosis for assessing disease outcome in a subject having COVID-19, the method comprising (a) receiving a biological sample obtained from the subject, the biological sample including RNA of the subject, (b) measuring, in the biological sample, a first gene expression level of a first human gene selected from the group consisting of genes shown in Table 12 or Table 13, (c) determining a classification of disease outcome using the first gene expression level and one or more reference gene expression levels determined from a plurality of reference samples having a known COVID-19 disease outcome classification. Variations of the method may further include measuring, in the biological sample, a second gene expression level of a second human gene selected from the group consisting of genes shown in Table 12 or Table 13, and determining a classification of COVID-19 disease outcome using the first gene expression level, the second gene expression level, and one or more reference gene expression levels determined from a plurality of reference samples having a known COVID-19 disease outcome classification.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows a hierarchal clustering of the patients based on the row Z-scores, with each cluster corresponding to the unique host transcriptional signature of SARS-COV-2 infection, other viral infection, or non-viral acute respiratory infection. The row Z-scores are shown broken into three groups for each of the categories where each of the nine groups show similar Z-scores. Generally, the “No Virus” subjects are on the left, the “SARS-COV-2” subjects are in the middle, and the “other Virus” subjects are on the right. The figure is found in Mick et al. “Upper airway gene expression reveals suppressed immune responses to SARS-CoV-2 compared with other respiratory viruses”. Nat Commun 11, 5854 (2020). doi.org/10.1038/s41467-020-19587-y, which is incorporated herein by reference for all purposes.

FIG. 2A shows receiver operating characteristic (ROC) curves for a 10-gene classifier. FIG. 2B shows ROC curves for a 3-gene classifier. Both classifiers were developed to differentiate COVID-19 from other acute respiratory illnesses (viral and non-viral) from the 234 subjects with expression profiles in FIG. 1. FIGS. 2A and 2B are from Mick et al., 2020, supra.

FIG. 3A shows the distribution of normalized, standardized gene counts for IFI6 across the entire filtered combined dataset. The plot shows IFI6 expression levels for SARS-COV-2 positive (SARS-COV-2+) and SARS-COV-2 negative (SARS-COV-2−) patients.

FIGS. 3B and 3C show scatterplots illustrating the distribution of samples in the filtered combined dataset based on counts for the two-gene set including (IFI6 and GBP5) for SARS-COV-2 positive (SARS-COV-2+) and SARS-COV-2 negative (SARS-COV-2−) patients.

FIG. 3D shows the distribution of normalized, standardized gene counts for GBP5 across the entire filtered combined dataset. The plot shows GBP5 expression levels for SARS-COV-2 positive (SARS-COV-2+) and SARS-COV-2 negative (SARS-COV-2−) patients.

FIG. 4A shows the distribution of normalized, standardized gene counts for IFI6 across the entire filtered combined dataset. The plot shows IFI6 expression levels for SARS-COV-2+, SARS-COV-2−, and patients with other virus infections.

FIGS. 4B and 4C show scatterplots illustrating the distribution of samples in the filtered combined dataset based on counts for the two-gene set including IFI6 and GBP5 for SARS-COV-2+, SARS-COV-2−, and patients with other virus infections.

FIG. 4D shows the distribution of normalized, standardized gene counts for GBP5 across the entire filtered combined dataset. The plot shows GBP5 expression levels for SARS-COV-2+, SARS-COV-2−, and patients with other virus infections.

FIG. 5 shows the area under the receiver operating characteristic ROC curve (AUC) for the diagnostic classifier with the two-gene set of IFI6 and GBP5, and a combined host-viral diagnostic classifier with the two-gene set of IFI6 and GBP5 and viral load (SARS-COV-2 reads per million (SARS-COV-2 rpM)) measure.

FIG. 6 shows AUC of single genes plotted as a function of mean log fold change illustrating performance of single genes to differentiate COVID-19 from non-viral acute respiratory illnesses when incorporating fold-change.

FIG. 7 shows AUC of genes paired with IFI6 plotted as a function of mean log fold change illustrating performance to differentiate COVID-19 from non-viral acute respiratory illnesses when incorporating fold-change.

FIG. 8 shows AUC of genes paired with IFI44 plotted as a function of mean log fold change illustrating performance to differentiate COVID-19 from non-viral acute respiratory illnesses when incorporating fold-change.

FIG. 9 shows a flowchart illustrating a method for performing a COVID-19 diagnosis, according to an embodiment.

FIG. 10 shows a flowchart illustrating a greedy feature selection algorithm for the identification of gene sets.

FIG. 11 illustrates a measurement system according to an embodiment of the present disclosure.

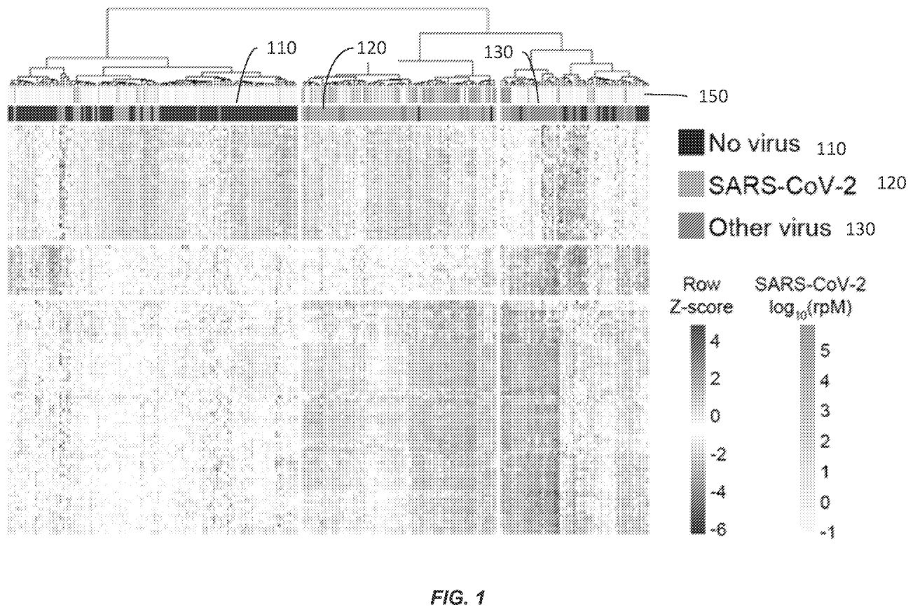

FIGS. 1, 2A, 2B, 3A, 3B, 3C, 3D, 4A, 4B, 4C, 4D, 5, 6, 7 and 8 comprise color features.

FIG. 12 shows a block diagram of an example computer system usable with systems and methods according to embodiments of the present disclosure.

### LIST OF TABLES

Table 1 shows lasso-selected features and coefficients of a 10-gene host classifier and a 3-gene host classifier.

Table 2 shows AUC scores for best-performing two-gene sets identified based on the filtered combined dataset.

Table 3 shows AUC scores for best-performing two-gene sets identified based on the unfiltered combined dataset.

Table 4 shows AUC scores for best-performing single gene and three-gene sets (with rpM included in the model) identified based on the unfiltered combined dataset.

Table 5 shows AUC scores for two-gene sets (as shown in Table 1) evaluated on the Ramlall et al. dataset (Nature Medicine, 2020; 26: 1609-1615).

Table 6 shows AUC scores for best-performing two-gene sets identified based on the filtered combined dataset (without scaling and centering).

Table 7 shows a ranked list of first genes and AUC scores from the combined dataset and the Ramlall et al. dataset.

Table 8 shows AUC scores and log fold-change (log FC) for best-performing single genes identified to differentiate COVID-19 from non-viral acute respiratory illnesses.

Table 9 shows a ranked list of second genes and AUC scores from the combined dataset.

Table 10 shows a ranked list of second genes and AUC scores from the Ramlall et al. dataset.

Table 11 shows host gene pairs identified to perform best for PCR or LAMP based diagnostic platforms.

Table 12A shows annotations for first genes (GROUP 1).

Table 12B shows annotations for second genes. (GROUP 1).

Table 12C shows exemplary two-gene sets (GROUP 1)

Table 13A shows the top 4 best performing first genes optimized for PCR or LAMP based diagnostic platforms with best performance for COVID-19 diagnosis based on a combination of AUC and fold change differences in expression.

Table 13B shows the top 8 best performing 2nd gene combinations, with examples based on IFI6 and IFI44 as the first gene.

Table 13C shows exemplary two-gene sets.

Table 13D shows annotations for four second genes not included in Table 12B.

Table 14 shows exemplary single genes and three-gene sets for use in a combined host-viral diagnostic assay.

### Terms

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated. The term “based on” is intended to mean “based at least in part on.”

A “biological sample” or “sample,” as used herein, generally refers to a substance obtained from a subject, e.g., a human subject. A biological sample contains analytes for example those described herein, i.e., nucleic acids, such as human RNA expressed by cells of the subject and potentially viral RNA from SARS-COV-2. In some embodiments, a biological sample is a sample comprising cells from the nose, mouth, throat or lower respiratory tract of the subject. A sample from the nose or mouth may be collected, for example, by a buccal swab, nasal swab, nasopharyngeal swab, nasopharyngeal wash or aspirate, mid-turbinate nasal swab, oropharyngeal swab, or saliva specimen. In some embodiments, the biological sample is a sample comprising fluid from the lungs, such as a broncho-alveolar lavage, or an endotracheal aspirate. In one embodiment, the biological sample is a sample comprising cells from the nose and is collected with a nasal swab. In one embodiment, the biological sample is a sample comprising cells from the nose and is collected with a nasopharyngeal swab. In one embodiment, the biological sample is a sample comprising cells from the throat and is collected with an oropharyngeal swab. In some embodiments, solid tissues, for example lung tissues, may be used as biological samples. Additional biological samples include include serum, plasma, or blood.

The term “diagnosing COVID-19” or “diagnosis of COVID-19” means to determine the presence or absence of a respiratory disease caused by a SARS-COV-2 infection in a subject. Thus, the term encompasses identifying in a subject with a respiratory illness whether the disease is caused by SARS-COV-2.

The term “viral RNA” refers to RNA with a sequence encoded by a viral genome (e.g., a nucleotide sequence of the SARS-COV-2 RNA), including fragments thereof and polymorphic sequences derived thereof.

The term “subject” or “patient” refers to an animal, such as a mammal. For example, a subject can be a human. In various examples, a subject can be healthy, or diagnosed or suspected of having a disease, such as an acute respiratory illness. In various examples, the disease may be COVID-19.

The term “nucleic acid” or “polynucleotide” refers to a deoxyribonucleic acid (DNA) or ribonucleic acid (RNA) and a polymer thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. It will be appreciated that, disclosure of a particular nucleic acid sequence is also a disclosure of the corresponding complementary nucleotide sequence, and that complementary sequences may be used in PCR assays, LAMP assays and other assays. Unless otherwise indicated, a particular nucleic acid sequence also implicitly encompasses conservatively modified variants thereof (e.g., degenerate codon substitutions) and/or alleles, and/or orthologs, and/or single nucleotide polymorphisms (SNPs), and/or copy number variants, as well as the sequence explicitly indicated.

The term “gene” means the segment of DNA involved in producing a polypeptide chain or transcribed RNA product. It may include regions preceding and following the coding region (leader and trailer) as well as intervening sequences (introns) between individual coding segments (exons).

As used herein, references to detecting, quantitating, measuring, or evaluating host “genes” (e.g., a pair comprising a first host gene and a second host gene) are understood to refer to detecting, quantitating measuring, or evaluating the expression level of the name gene, typically by quantitating RNA transcribed from the gene, typically using RT-PCR or RT-LAMP.

The term “SARS-CoV-2 viral gene” means the segment of SARS-COV-2 viral RNA involved in producing a polypeptide chain (e.g., a SARS-COV-2 protein).

The terms “host marker” or “host gene marker”, as used herein, refer to diagnostic indicators found in a subject and which may be used for diagnosis, prognosis, or other classification purposes. Such markers can be identified by embodiments of the present disclosure. The host gene marker refers to an entire gene (or a portion thereof) from the subject (as opposed to the virus) that has been found to show differential expression in the subject when infected with COVID-19. In other examples, a gene marker may be a prognostic indicator found in a subject, the expression or level of which changes between certain conditions of disease outcome (e.g., mild versus severe illness).

The terms “viral marker” or “viral gene marker”, as used herein, refer to a nucleotide sequence of SARS-COV-2 which can be used as an indicator for the presence of the virus in a biological sample. In particular, the viral marker may be an entire nucleotide sequence or portion thereof that encodes a SARS-COV-2 genome. The nucleotide sequence of a SARS-COV-2 genome may differ from strain to strain. An example of a SARS-COV-2 genome includes the Wuhan-Hu-1 genome (Genbank Accession No. MN908947.3). The RNA genome of SARS-COV-2 virus is about 29.8 kb to 30 kb in length and encodes at least 29 proteins (“SARS-COV-2 proteins”) including four structural proteins, at least 16 predicted non-structural proteins, and several accessory proteins. In some embodiments, a gene encoding SARS-COV-2 structural protein Nucleocapsid (N) or a gene encoding SARS-COV-2 structural protein Envelope (E) may be used as a viral marker.

As used herein, “SARS-COV-2” refers to a positive-strand, or “sense-strand,” RNA virus that causes COVID-19.

“Signs and symptoms” of COVID-19 disease include fever or chills; cough; shortness of breath/difficulty breathing; fatigue; runny or stuffy nose; muscle or body aches; headache; sore throat; nausea or vomiting; diarrhea; new loss of taste or smell; persistent pain or pressure in the chest; new confusion; inability to wake up or stay awake; bluish lips or face, and acute respiratory illness. Other signs and symptoms will be recognized by medical professionals.

As used herein, the term “acute respiratory illness,” or “ARI” refers to an illness affecting the upper and/or lower respiratory tract. Typical symptoms include, for example, coughing, wheezing, fever, sore throat, and congestion. Physical findings may include elevated heart rate, elevated breath rate, abnormal white blood cell count, and low arterial carbon dioxide tension. ARIs are often caused by viral or bacterial pathogens, and are characterized by rapid progression of symptoms over hours to days. ARIs may primarily be of the upper respiratory tract, the lower respiratory tract, or a combination of the two. ARIs may have systemic effects due to spread of the pathogen beyond the respiratory tract or due to collateral damage induced by the immune response.

As used herein, the term “diagnose” has its normal meaning. In one approach, without limitation, a subject is diagnosed as having COVID-19 disease by (i) determining that the subject is infected with SARS-COV-2 and (ii) determining that the subject exhibits signs or symptoms characteristic of COVID-19 disease (such as acute respiratory illness).

As used herein, the term “differentially expressed” refers to differences in the expression level or abundance (i.e., in the quantity and/or the frequency) of a gene marker (e.g., RNA) present in a sample taken from patients having COVID-19 as compared to a reference sample, e.g., obtained from a subject not infected with COVID-19 or a subject with different disease manifestation. For example, the transcript or RNA levels of a gene marker may be present at an elevated level or at a decreased level in samples of patients with COVID-19 compared to reference samples. In another example, the RNA levels of a gene marker may be present at an elevated level or at a decreased level in samples of patients with severe COVID-19 compared to reference samples obtained from subjects having mild COVID-19.

The term “reference sample,” as used herein, refers to a sample having a known state (e.g., COVID-19 classification or outcome classification). Gene expression in the reference sample may be used as a baseline or reference value with which to compare expression in a test sample. In particular, the expression level of a corresponding gene from the reference sample (herein referred to as a “reference gene expression level”) can be used to compare against the sample for which a classification is to be determined. In various examples, reference gene expression levels from a training set of reference samples may be used to generate a diagnostic or prognostic classifier. A reference sample can be a sample obtained from a healthy subject or populations of healthy subjects, i.e., a subject who does not have symptoms or signs of acute respiratory illness, is not infected with SARS-COV-2, and who does not have COVID-19. In some embodiments, a reference sample is a sample obtained from an infected subject having COVID-19. In various examples, a reference sample may be obtained from a subject of known disease phenotype outcome. For example, reference samples may be obtained from subjects with mild disease manifestation and/or from subjects with severe disease manifestation. In some embodiments, reference samples may be from subjects with different COVID-19 disease outcomes. For example, reference samples may be used from subjects that develop acute respiratory distress syndrome (ARDS) and/or respiratory failure requiring mechanical ventilation. In some embodiments, reference samples may be used from subjects that show a certain response to a certain therapy or treatment. For example, reference samples may be from subjects that do not develop ARDS and/or respiratory failure requiring mechanical ventilation in response to a certain therapy or treatment. In some embodiments, reference samples may be from subjects that had a 30-day mortality. In some embodiments, methods of diagnosing COVID-19 in a human subject involve using values obtained from reference subjects who are age and/or gender matched with the subject.

The terms “identical” or percent “identity,” in the context of two or more nucleic acids or polypeptide sequences, refer to two or more sequences or subsequences that are the same (“identical”) or have a specified percentage of amino acid residues or nucleotides that are the same (i.e., at least about 70% identity, at least about 75% identity, at least 80% identity, at least about 90% identity, preferably at least about 91%, 92%, 93%, 94%, 95%, 96%, 97%, 98%, 99%, or higher identity over the entire sequence of a specified region, when compared and aligned for maximum correspondence over a comparison window or designated region. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (www.ncbi.nlm.nih.gov/).

The term “classification” as used herein refers to any number(s) or other characters(s) that are associated with a particular property of a subject. For example, a subject could be assigned to one or more categories or outcomes (e.g., a patient is infected or is not infected with SARS-COV-2, another categorization may be that a patient is infected with a viral ARI or infected with a non-viral ARI). In some cases, a “+” symbol (or the word “positive”) could signify that a sample is classified as having COVID-19 disease or a SARS-COV-2 infection. The classification can be binary (e.g., positive or negative) or have more levels of classification (e.g., a scale from 1 to 10 or 0 to 1), as may be done for different levels of disease manifestation. The outcome, or category, is determined by the value of the scores provided by the classifier, which may be compared to a cut-off or threshold value. The terms “cutoff” and “threshold” refer to predetermined numbers used in an operation. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different groups of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples.

An “Entrez Gene ID” number identifies a gene entry in the National Center for Biotechnology Information (NCBI) gene database (www.ncbi.nlm.nih.gov/gene/). An “HGNC ID” number identifies a gene entry in the HGNC database (www.genenames.org). The phrase “a sequence listed in Table X” means “a sequence of a gene identified by an Entrez Gene ID or a HGNC ID listed in Table X.”

### DETAILED DESCRIPTION

Coronavirus disease 2019 (COVID-19) is a respiratory illness caused by severe acute respiratory syndrome coronavirus 2 (SARS-COV-2). Symptoms of COVID-19 are variable, but often include fever, cough, fatigue, breathing difficulties, and loss of smell and taste. While most patients have mild symptoms, some people develop severe disease that requires hospitalization and oxygen support, and some require admission to an intensive care unit. In severe cases, COVID-19 can be complicated by the acute respiratory distress syndrome (ARDS).

The standard method of testing for presence of SARS-COV-2 is real-time reverse transcription polymerase chain reaction (rRT-PCR; Kilic et al. (2020), “Molecular and immunological diagnostic tests of COVID-19: Current status and challenges, iScience, 23(8): 101406; Coronavirus disease (COVID-19) technical guidance: Laboratory testing for 2019-nCoV in humans,” World Health Organization (WHO), Retrieved Dec. 7, 2020), which detects the presence of viral RNA. Viral targets are selected from, e.g., the E, N, S, and Orf1ab regions of SARS-COV-2 genome. However, insufficient viral loads or mutations in PCR primer target regions in the SARS-COV-2 genome contribute to false negative results.

### I. Analysis of Host Markers

Transcriptome analysis of host responses to virus infection can be used to reveal systemic changes in host gene expression profiles caused by the viral infection. By comparing such transcriptomic profiles in samples from subjects with the virus infection versus those without, it is possible to identify genes that differ in their expression between the groups, and thus are part of the disease signature. The transcriptional signatures can be used as diagnostic tools allowing the classification of individuals based on the expression profile of the identified gene markers.

FIG. 1 shows a host transcriptional response to SARS-COV-2 assessed by upper airway metagenomic RNA sequencing (using oropharyngeal (OP) or nasopharyngeal (NP) swabs) from a cohort of patients having COVID-19 (n=93), other viral (n=41), or non-viral (n=100) acute respiratory illnesses (see Mick et al. (n=234; “Upper airway gene expression reveals suppressed immune responses to SARS-COV-2 compared with other respiratory viruses,” Nature Communications 2020, 11: 5854). In FIG. 1, the columns correspond to different patients. The different patient groups are labeled as “No Virus” 110, “SARS-COV-2” 120, and “other Virus” 130. The rows correspond to different genes, with the row Z-score corresponding to a differential expression level for the gene relative to the expression level for a patient with no virus. The Z-score is determined by taking a difference between the measured expression level for a given patient and an average expression level for the “no virus” patients, and then dividing by a standard deviation in the measurements for the “no virus” patients.

The bar 150 shows the viral load intensity of a marker for SARS-COV-2 as measured by mNGS reads per million (rpM). All samples were processed through a SARS-COV-2 reference-based assembly pipeline that involved removing non-SARS-COV-2 reads with Kraken2 (Wood & Langmead (2019), “Improved metagenomic analysis with Kraken 2,” Genome Biol. 20, 257), and aligning to the SARS-COV-2 reference genome MN908947.3 using minimap2 (U at al. (2018), “Minimap2: pairwise alignment for nucleotide sequences,” bioinformatics, 34, 3094-3100). We calculated SARS-COV-2 reads-per-million (rpM) using the number of reads that aligned with mapq >=20. For plotting purposes, 0.1 was added to the rpM values to avoid taking the log of 0. As can be seen, the subjects in the category “SARS-COV-2” 120 generally have the highest intensity of the marker for SARS-COV-2.

Using the data shown in FIG. 1, we designed and built 10- and 3-gene host classifiers using random forest and regularized regression machine learning (Table 1). For example, for feature selection, we used the logistic lasso (as implemented in the R package glmnet), and then trained random forests on the selected features (using the R package randomForest). The results of this classification approach provide information on the genes that afford the best discrimination between the different patient categories. Both classifiers were developed to differentiate COVID-19 from other acute respiratory illnesses (viral and non-viral). The values in Table 1 are the coefficients that multiply the expression levels for each gene, where the results are used in a random forest model. In Table 1, the intercept corresponds to the intercept (vertical offset) for the linear regression function used for logistic regression.

TABLE 1
Lasso-selected features and coefficients
of 10- and 3-gene host classifiers.
10-gene model

| | |
|---|---|
| (Intercept) | −4.733 |
| PCSK5 | 0.046 |
| IL1R2 | −0.055 |
| IL1B | −0.041 |
| IFI6 | 0.452 |
| WDR74 | 0.124 |
| FAM83A | 0.004 |
| ADM | −0.099 |
| IFI27 | 0.084 |
| KRT13 | −0.005 |
| DCUN1D3 | −0.05 |

3-gene model

| | |
|---|---|
| (Intercept) | −2.808 |
| IL1R2 | −0.037 |
| IL1B | −0.052 |
| IFI6 | 0.372 |

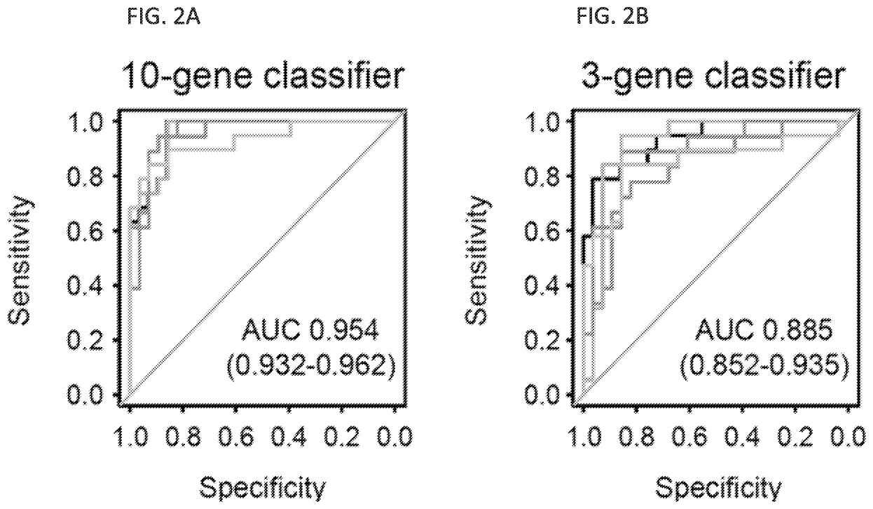

FIG. 2A shows receiver operating characteristic (ROC) curves for the 10-gene classifier. FIG. 2B shows ROC curves for the 3-gene classifier. These classifiers accurately identified patients with COVID-19 with an area under the receiving operator characteristic (ROC) curve of 0.954 and 0.885, respectively. The different lines correspond to different classifiers. The range in AUC and the average AUC is provided. The classifiers in Table 1 are ones with the highest AUC.

### II. Use of Viral and Human Expression Levels

High-throughput RNA sequencing (RNA-seq) technology, combined with bioinformatics has emerged as a powerful approach for the discovery of host gene signatures. As described above, host transcriptional profiling of respiratory specimens is an accurate and sensitive approach for investigating differential gene expression that arises in response to disease (e.g., COVID-19). We now extend these methods by simultaneously profiling host and viral markers in a novel assay designed to improve detection of COVID-19 disease and/or SARS-COV-2 infection and predict outcomes. In particular, we used metagenomic next generation RNA-sequencing (mNGS) which can simultaneously identify airway pathogens and the host transcriptome, thus allowing comprehensive evaluations of disease pathophysiology and prediction of clinical outcomes. mNGS involves sequencing the nucleic acids in a sample to analyze the reads generated by aligning reads to the host genome and reference databases, such as the National Center for Biotechnology Information (NCBI) GenBank database. Host gene expression can be quantified by counting the number of reads that mapped to each locus in the genome.

Here, we address the need for an improved COVID-19 diagnostic assay that simultaneously detects SARS-COV-2 and transcriptional gene markers of the host's immune response. An assay that integrates host response and virus detection could improve diagnostic accuracy and more precisely inform optimal antiviral treatment. Importantly, the gene marker panel and the methods provided herein are well suited to be used with existing protocols, such as polymerase chain reaction (PCR) or loop-mediated isothermal amplification (LAMP) based assays.

The present invention is based, at least in part, on the discovery that the expression level of certain host genes in combination with the expression profile of viral markers can predict whether a subject has COVID-19. In particular, we have discovered that the measured expression levels of just two or three host genes alone or in combination with the measured expression levels of a viral marker can be used too as diagnostic markers of COVID-19. In one approach, the diagnostic assay provided herein can be used to diagnose a SARS-COV-2 infection. As described in the examples below, a diagnostic assay including host gene expression levels is more accurate in distinguishing between SARS-COV-2+ and SARS-COV-2− samples. In one approach, the diagnostic assay provided herein can be used to diagnose or identify disease, i.e., COVID-19. Specifically, the diagnostic assay provided herein allows to distinguish COVID-19 from other respiratory illnesses (viral or non-viral).

We used mNGS and data analysis to profile gene expression using nasopharyngeal (NP) with or without pooling with an oropharyngeal (OP) swab collected from cohorts of subjects with COVID-19, other viral ARIs or non-viral ARIs. Using a machine learning-based approach, we identified a gene panel and gene subsets that alone or in combination with viral markers (e.g., SARS-COV-2 RNA) can accurately differentiate COVID-19 from other viral ARIs and/or non-viral ARIs. Thus, provided herein are host-based diagnostic method for classifying individuals based on the expression levels of the identified gene markers.

The following sections describe the generation of a COVID-19 classifier using host and viral expression levels, as well as results for different gene panels.

### III. Generating a Classifier for Covid-19

Classifiers that use viral and host gene expression levels can be generated from a training set of samples obtained from patients having known classifications, e.g., for diagnosis or prognosis. Measurements of many viral and host genes can be obtained. The measurements can be analyzed to determine set of genes (i.e., their expression levels) that best discriminate between the different classifications of the training set via an optimization procedure. As an example, embodiments can generate a diagnostic classifier for COVID-19, by (i) receiving a biological sample from a plurality of subjects having a known diagnosis, (ii) measuring gene expression levels of a plurality of genes to determine the transcriptional profile of these training samples, and (iii) analyzing the gene expression data to identify COVID-19 associated gene expression signatures that distinguish COVID-19 subjects from other subjects, thereby generating a diagnostic classifier for COVID-19.

The analysis of gene expression data can include training a machine learning model to distinguish between positive and negative samples based on the expression level of certain genes. The analysis can include using the gene expression data as a training set where the gene expression levels (acting as input features to the model) and known diagnosis (labels) are used to train a machine learning model to distinguish between positive and negative samples (or between COVID-19 and other diseases caused by viral infections e.g., other viral ARI infections). In the process of learning, the model identifies gene markers that are predictive for the disease state.

In some embodiments, different subsets of genes can be selected to form a subset of training samples. This training subset can then be used to train (optimize) a model, whose accuracy can be measured, e.g., using the AUC of an ROC curve. Then, another subset of genes can be selected, with a further training process providing another model whose accuracy can also be measured. The accuracy can be measured using the training set or a validation set, which can include samples with known labels that were excluded from the training set. This process of generating models for different subsets of genes, along with the accuracy of each model, can continue, possibly for all possible subsets of genes for which expression levels have been measured. The subsets can be constrained to a specified number of host genes (e.g., 1 or 2) and a specified number of SARS-COV-2 viral genes (e.g., 1).

To generate the training data, RNASeq gene expression data were obtained from multiple patients across three cohorts along with an indication of whether each patient was diagnosed as SARS-COV-2 positive (SARS-COV-2+) or SARS-COV-2 negative (SARS-COV-2−). For SARS-COV-2− patients we further had an indication of whether the acute respiratory illness was caused by a virus other than SARS-COV-2 (“Other Virus”) or was not caused by any virus (“No Virus”) based on clinical PCR or metagenomic sequencing. The three cohorts used in the work described below were: the dataset from Mick et al. (n=234; “Upper airway gene expression reveals suppressed immune responses to SARS-COV-2 compared with other respiratory viruses,” Mick et al., Nature Communications 2020, 11: 5854, a dataset from UCSF (n=130), and a dataset from Ramlall et al. (n=553; Immune complement and coagulation dysfunction in adverse outcomes of SARS-COV-2 infection,” Ramlall et al., Nature Medicine 2020, 26: 1609-1615). The Mick et al. dataset was combined with the UCSF dataset, forming a pooled dataset referred to as the “combined dataset.”

The patients included in the cohorts presented with one or more of a fever, cough, shortness of breath, headache, nasal congestion, rhinorrhea, sore throat, loss of smell, and unexplained muscle aches. Patients were assessed by a physician in an outpatient, inpatient or emergency department setting. We selected for patients early in the disease course by filtering the patients based on the Cycle threshold (Ct) as described in section VII.A. We then used the gene expression data to construct a classifier capable of accurately differentiating COVID-19 from other ARIs (viral or non-viral). The dataset was randomly split into a training set (70% of samples) and a validation set (30% of samples). As described in more detail below, the training set was used for identifying n-gene sets, and both cohorts (along with the Ramlall et al. dataset) were used for evaluating the performance of the n-gene sets. By employing a greedy feature selection algorithm (see section VII.B.1), we identified host genes and get sets that alone or in combination with a viral marker can determine whether a subject has COVID-19.

As described above, we used cohorts consisting of patients that were diagnosed as SARS-COV-2+ or SARS-COV-2−. In various examples, other cohorts may be used. Any suitable cohort may be used depending on the classification type. For example, for the generation of a prognostic classifier for COVID-19 a cohort may be used for which the disease outcome is known for each sample (such as disease severity, development of respiratory failure requiring mechanical ventilation, development of acute respiratory distress syndrome (ARDS), duration of hospitalization, response to a treatment, and/or mortality, e.g., 30 day mortality). In some examples, biological samples are obtained from healthy individuals and individuals with COVID-19. In some examples, biological samples are obtained from subjects having COVID-19 with different disease outcomes.

Exemplary biological samples are described herein and include those obtained, for example, by a nasal swab, nasopharyngeal swab, nasopharyngeal wash or aspirate, mid-turbinate nasal swab, oropharyngeal swab, buccal swab, a broncho-alveolar lavage, or an endotracheal aspirate. In some embodiments, the biological sample is serum, plasma, blood, or solid tissue. The biological sample includes RNA of the subject and potentially RNA from SARS-COV-2. In some embodiments, a sample may be processed to provide or purify RNA or a particular nucleic acid molecule or fragment thereof.

Gene expression levels may be determined using any suitable method. For example, RNA may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing. Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR). Gene expression may also be assessed by PCR, Loop-Mediated Isothermal Amplification (LAMP), Transcription-Mediated Amplification (TMA), Isothermal Amplification or other nucleic acid amplification assay.

The machine learning model (or more generally model) can be trained using the gene expression data with the corresponding labels (known diagnostic outcome) as a training data set. Generally, the machine learning model is a collection of parameters and functions (as detailed in section VII), where the parameters are trained using the training data set. Training data sets may be selected by random sampling of a set of data corresponding to one or more sets of subjects (e.g., retrospective and/or prospective cohorts of patients having or not having COVID-19). Alternatively, training data sets may be selected by proportionate sampling of a set of data corresponding to one or more sets of subjects (e.g., retrospective and/or prospective cohorts of patients having or not having COVID-19). Training sets may be balanced across sets of data corresponding to one or more sets of subjects (e.g., patients from different clinical sites or trials).

In some embodiments, the model is trained to distinguish between a SARS-COV-2+ or SARS-COV-2− sample. Other examples may include different models, each one directed to a different type of classification. For example, a model can determine whether a subject having COVID-19 will have a mild or severe illness. A further model can determine whether a subject having COVID-19 will develop ARDS not. A further model can determine whether the subject has an increased mortality risk or not. A further model can classify a predicted response of a subject to a particular type of treatment.

The machine learning model may be trained until certain predetermined conditions for accuracy or performance are satisfied, such as having minimum desired values corresponding to diagnostic accuracy measures. For example, the diagnostic accuracy measure may correspond to prediction of a diagnosis or disease outcome in the subject. Examples of diagnostic accuracy measures may include sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting COVID-19.

### IV. Results for Different Gene Panels

Using the methods described above for classifier generation, we identified several gene sets capable of accurately differentiating COVID-19 from other ARIs (viral or non-viral). In the following sections we summarize the results by showing which genes were identified for each of the models and how each of these gene sets performed as diagnostic classifiers.

### A. Two-Gene Sets

In this section we focus on the host's immune response to SARS-COV-2 infection and/or COVID-19 and present results based on the host gene expression only, i.e., without assessing viral load. We show which gene sets were identified and summarize the performance of the host transcriptional classifiers that alone (i.e., without viral markers) can differentiate COVID-19 from other ARIs. We specifically focus on gene sets comprising a small number of genes (e.g., two gene sets) in order to allow the gene panel to be used in existing platforms (e.g., PCR, LAMP or TMA platforms) that are designed to detect and measure expression levels of a few targets only. For example, existing platforms that are designed to detect three targets (i.e., three viral markers) could be repurposed to detect host genes instead (or a combination of host genes and viral marker, see section IV.B, below).

By employing a modified version of the greedy feature selection algorithm (see Section VII.B.1), we identified nine two-gene sets capable of accurately differentiating COVID-19 from other ARIs (viral or non-viral). The results using the combined dataset filtered for samples with Cycle threshold (Ct)<30 (referred to as the “filtered combined dataset” and as described in section VII.A) are shown in Table 2 (columns labelled “Without rpM”, i.e., without viral load (SARS-COV-2 reads per million (rpM) measure). The first column of Table 2 shows average AUC scores calculated on 10000 rounds of 5-fold cross-validation (CV) on the 70% training sample. The second column shows AUC scores calculated on 10000 rounds of 5-fold cross-validation on the 30% validation sample. The third column shows AUC scores generated by testing the 30% validation sample on models each trained on the 70% training sample.

TABLE 2
Best Performing 2-Gene Sets on Combined Dataset Filtered for Samples
with Ct <30. Numbers in parentheses are standard deviations.

| | Without rpM | | | With rpM | |
|---|---|---|---|---|---|
| | 70% Training | 30% Validation | 30% Validation | 70% Training | 30% Validation |
| | Set (n = 222, | Set (n = 96, | Set (n = 96, | Set (n = 222, | Set (n = 96, |
| | 5-fold CV, | 5-fold CV, | trained on 70% | 5-fold CV, | trained on 70% |
| Gene Set | 10000 rounds) | 10000 rounds) | training set) | 10000 rounds) | training cohort) |
| IFI6, PTAFR | 0.960 (0.004) | 0.880 (0.017) | 0.858 | 0.999 (0.001) | 0.966 |
| IFI6, GBP5 | 0.955 (0.004) | 0.910 (0.014) | 0.868 | 0.999 (0.001) | 0.974 |
| IFI6, GRINA | 0.954 (0.004) | 0.916 (0.014) | 0.896 | 0.999 (0.001) | 0.966 |
| IFI44, TPM4 | 0.946 (0.004) | 0.837 (0.020) | 0.772 | 0.999 (0.0005) | 0.974 |
| IFI44, BAZ1A | 0.944 (0.004) | 0.889 (0.017) | 0.785 | 0.998 (0.001) | 0.974 |
| IFI44, VCAN | 0.944 (0.004) | 0.841 (0.018) | 0.806 | 0.998 (0.001) | 0.966 |
| IFI44L, GBP5 | 0.952 (0.004) | 0.865 (0.017) | 0.851 | 0.999 (0.0005) | 0.993 |
| IFI44L, BAZ1A | 0.947 (0.004) | 0.854 (0.019) | 0.824 | 0.998 (0.001) | 0.993 |
| IFI44L, SH3BP2 | 0.946 (0.004) | 0.857 (0.016) | 0.794 | 0.999 (0.001) | 0.979 |

We also performed the same evaluations using the unfiltered version of the combined dataset, i.e., not filtered for Ct<30 (see section VII.A). The results are shown in Table 3 (columns labelled “Without rpM”).

TABLE 3
Best Performing 2-Gene Sets on Combined Dataset Not Filtered for Samples
with Ct <30. Numbers in parentheses are standard deviations.

| | Without rpM | | | With rpM | |
|---|---|---|---|---|---|
| | 70% Training | 30% Validation | 30% Validation | 70% Training | 30% Validation |
| | Set (n = 257, | Set (n = 111, | Set (n = 111, | Set (n = 257, | Set (n = 111, |
| | 5-fold CV, | 5-fold CV, | trained on 70% | 5-fold CV, | trained on 70% |
| Gene Set | 10000 rounds) | 10000 rounds) | training set) | 10000 rounds) | training set) |
| IFI6, PTAFR | 0.860 (0.008) | 0.854 (0.013) | 0.869 | 0.869 (0.007) | 0.924 |
| IFI6, GBP5 | 0.887 (0.005) | 0.876 (0.012) | 0.895 | 0.879 (0.007) | 0.929 |
| IFI6, GRINA | 0.853 (0.008) | 0.902 (0.011) | 0.899 | 0.884 (0.007) | 0.940 |
| IFI44, TPM4 | 0.865 (0.005) | 0.838 (0.013) | 0.850 | 0.913 (0.006) | 0.918 |
| IFI44, BAZ1A | 0.855 (0.006) | 0.867 (0.013) | 0.899 | 0.867 (0.010) | 0.914 |
| IFI44, VCAN | 0.841 (0.009) | 0.792 (0.014) | 0.829 | 0.858 (0.009) | 0.884 |
| IFI44L, GBP5 | 0.879 (0.005) | 0.852 (0.012) | 0.880 | 0.884 (0.009) | 0.928 |
| IFI44L, BAZ1A | 0.871 (0.006) | 0.824 (0.013) | 0.864 | 0.873 (0.011) | 0.907 |
| IFI44L, SH3BP2 | 0.868 (0.006) | 0.848 (0.013) | 0.863 | 0.883 (0.008) | 0.888 |

One of the advantages of looking at two-gene models, as opposed to models that incorporate larger numbers of features, is that the models' operation can be visualized by creating two-dimensional plots comparing the expression of the genes.

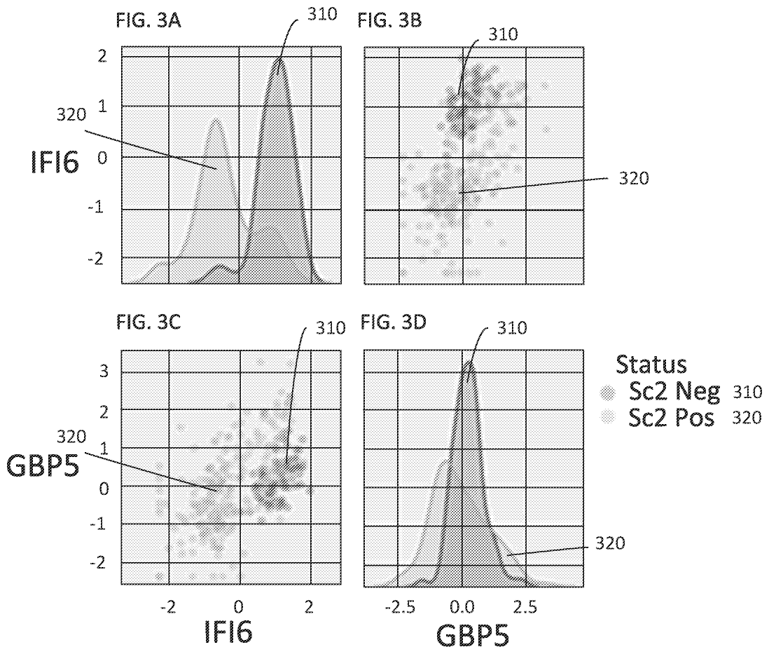

FIGS. 3A-D shows plots for one of the two gene sets, IFI6 and GBP5. FIGS. 3A-3D show each of the two patient groups in the filtered combined dataset: The different patient groups are labeled as “Sc2 Neg” 310 (SARS-COV-2−) and “Sc2 Pos” 320 (SARS-COV-2+). FIG. 3A shows the distribution of gene counts for IFI6 across the entire filtered combined dataset. The x-axis represents normalized, standardized gene counts (see section VII.A) for IFI6 and the y-axis represents the density. FIG. 3D shows the distribution of gene counts for GBP5 across the entire filtered combined dataset. The x-axis represents normalized, standardized gene counts (see section VII.A) for GBP5 and the y-axis represents the density. FIGS. 3B and 3C are scatterplots showing the distribution of samples in the filtered combined dataset based on counts for both genes. The two scatterplots (FIGS. 3B and 3C) are identical, just with the x and y axis swapped. FIG. 3A-3D show that the combination of IFI6 and GBP5 is effective at separating SARS-COV-2+ samples and SARS-COV-2− samples.

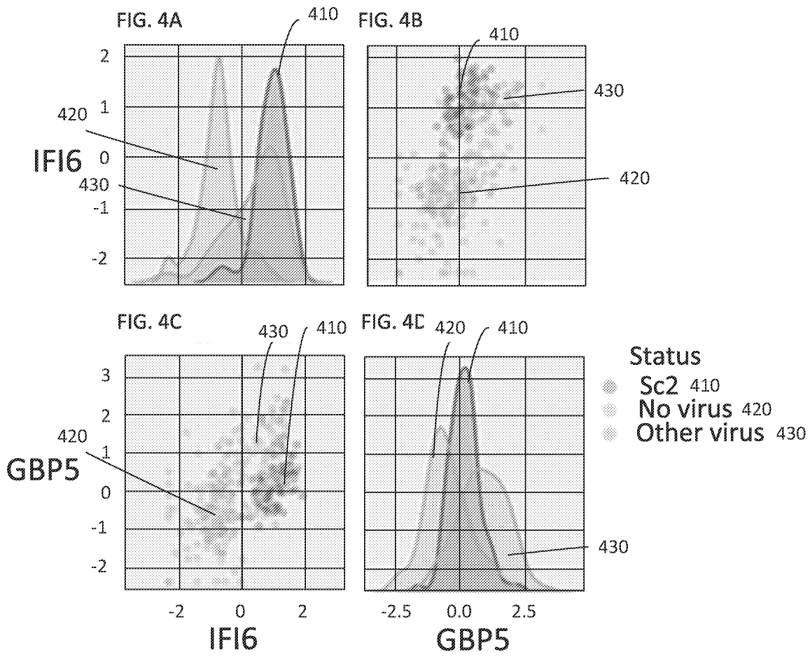

FIGS. 4A-4D show each of the three patient groups in the filtered combined dataset (SARS-COV-2+, no virus and other virus). The different patient groups are labeled as “Sc2” 410, “No virus” 420, and “Other virus” 430. FIG. 4A shows the distribution of gene counts for IFI6 across the entire filtered combined dataset. The x-axis represents normalized, standardized gene counts for IFI6 and the y-axis represents the density. FIG. 4D shows the distribution of gene counts for GBP5 across the entire filtered combined dataset. The x-axis represents normalized, standardized gene counts (see section VII.A) for GBP5 and the y-axis represents the density. FIGS. 4B and 4C are scatterplots showing the distribution of samples in the filtered combined dataset based on counts for both genes. The two scatterplots (FIGS. 4B and 4C) are identical, just with the x and y axis swapped. 4A-4D provide additional insight into how the two genes (IFI6 and GBP5) are working together. IFI6 alone almost completely separates the SARS-COV-2+ samples from “no virus” samples. However, many of the “other virus” samples have equivalent levels of IFI6 expression as the SARS-COV-2+ samples. Adding GBP5 to the model allows for separation of the SARS-COV-2+ samples from the “other virus” samples, and in fact the actual separation is better than might have been predicted based on the expression curves. Expression levels of GBP5 and IFI6 are somewhat correlated in the “other virus” samples, as can been seen in the fact that the dots in FIGS. 4B and 4C lie mostly on a diagonal stretching from the lower left to the upper right. Because of this, most of the samples that highly express IFI6 and therefore overlap the SARS-COV-2+ samples in IFI6 expression also highly express GBP5, which distinguishes them from the SARS-COV-2+ samples on this axis.

### B. Combined Host-Viral Gene Sets

The same two-genes sets were also evaluated in combination with SARS-COV-2 reads per million (rpM) values, which is a measurement of viral load as determined by counting the number of reads per million mapped reads. While rpM values are highly correlated with SARS-COV-2 positivity, they are not entirely predictive. And since one of the reasons for identifying host response genes is to augment detection methods that rely on the presence of viral sequences, we wanted to investigate how the two-gene sets performed when rpM values were included in the model as an additional feature.

AUC results for the filtered combined dataset when including rpM values in the model are shown in Table 2 (columns labelled “With rpM”). AUC results for the unfiltered combined dataset when including rpm values in the model are shown in Table 3 (columns labelled “With rpM”). The high AUC scores for the filtered dataset were expected, as the filtration process removed SARS-COV-2+ samples with low rpM counts. As a result, the SARS-COV-2+ and SARS-COV-2− populations in the filtered dataset are easily separated by rpM counts alone. For the unfiltered dataset, the inclusion of rpM as a feature increases the AUC scores for all genes, but the effects are rather modest. It is known that expression of some of these genes (e.g., IFI6—see Mick et al., 2020) is correlated with rpM levels, which might explain why adding rpM as a feature has only a small effect.

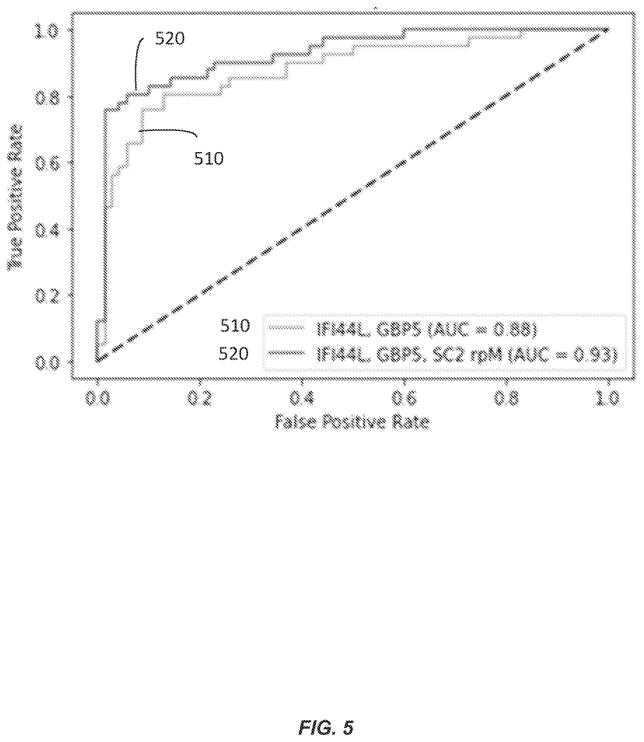

FIG. 5 shows that the host and viral diagnostic classifier for COVID-19 (IFI6, GBP5 and viral load (SARS-COV-2 rpM)) demonstrates enhanced performance when tested on RNAseq data. The figure shows the performance for a classifier including a two gene set alone and a classifier including the two gene sets and rpM values as an additional feature. The two classifiers are labeled as “IFI6, GBP5” 510 and “IFI6, GBP5, SARS-COV-2 rpM” 520. The performance of the classifier with viral load (IFI6, GBP5, SARS-COV-2 rpM) was enhanced with an AUC of 0.93 as compared to the classifier without viral load (IFI6, GBP5) that had an AUC of 0.88.

In another experiment, a greedy selection algorithm (See section VII.B.1) was applied to the unfiltered combined dataset and included rpM as a feature during the process, thereby selecting for genes that would perform well in combination with rpM. These gene sets are shown in Table 4. As expected, rpM values alone (first row of Table 4) produce a relatively high AUC score, but adding other genes such as WDR74 and PDGFRB enhances the ability of the model to distinguish between SC+ and SC−.

TABLE 4
Best Performing 1-Gene and 3-Gene Sets (with rpM) on Combined Dataset Not Filtered
for Samples with Ct <30. Numbers in parentheses are standard deviations.

| | 70% Training | 30% Validation | 30% Validation |
|---|---|---|---|
| | Set (n = 257, | Set (n = 111, | Cohort (n = 111, |
| Gene Set | 5-fold CV, | 5-fold CV, | trained on 70% |
| (+SARS-COV-2 rpM) | 10000 rounds) | 10000 rounds) | training set) |
| None (i.e., SARS-COV-2 rpM | 0.836 (0.014) | 0.872 (0.016) | 0.894 |
| alone) | | | |
| WDR74 | 0.908 (0.007) | 0.926 (0.016) | 0.948 |
| PDGFRB | 0.905 (0.005) | 0.942 (0.007) | 0.945 |
| WDR74, ATP13A4, OTUD7A | 0.964 (0.004) | 0.918 (0.011) | 0.930 |
| WDR74, PLK4, PDGFRB | 0.952 (0.004) | 0.968 (0.008) | 0.984 |
| WDR74, OR2C3, WASF1 | 0.958 (0.005) | 0.918 (0.010) | 0.924 |
| PDGFRB, PBDC1, POP1 | 0.966 (0.004) | 0.936 (0.009) | 0.952 |
| PDGFRB, POSTN, MMADHC | 0.962 (0.005) | 0.938 (0.011) | 0.971 |
| PDGFRB, ATP5F1C, GRIN2C | 0.911 (0.007) | 0.878 (0.014) | 0.951 |

### C. Testing on Independent Data Sets

To determine whether the two-gene sets remained predictive when applied to a completely independent dataset, support vector classifier (SVC; as described in section VII.B.1) models using each two-gene set were evaluated on the Ramlall et al. dataset. In one example, the Ramlall et al. dataset was used for training as well as validation; this is the cross-validation below. In another example, the Ramlall et al. dataset was just used for validation, with the training done using the combined dataset.

For the training and cross-validation using the Ramlall et al. dataset, average AUC scores and standard deviations were calculated after running 10,000 rounds of 5-fold cross-validation on the Ramlall et al. dataset. For each round, the Ramlall et al. dataset was split into 5 groups, with four groups used for training and the fifth group is used to validate the model, namely to determine an AUC score. Then, another four groups were used for training, and a different fifth group used for validation, and so on. Thus, five AUC values were determined for each round, and then averaged to determine the AUC for that round. The standard deviation (STD) is then determined using the AUC values for all rounds.

Each model was also trained on the 70% training set from the combined dataset and tested on the entire Ramlall et al. dataset.

Results are shown in Table 5 (columns labelled “Ramlall et al. Dataset”). The values for the 5-fold cross-validation are the average AUC score across the 10,000 rounds and the corresponding STD value. Other columns show performance of the two-gene sets on the combined dataset, which are duplicates of the first 3 data columns in Table 2 and are included here to allow comparison to the Ramlall et al. dataset. Overall, the identified gene sets performed well on the Ramlall et al. dataset, even when trained on the 70% training set of the combined dataset, demonstrating that these gene sets are able to generalize across models. It should be noted that achieving these scores required that the Ramlall et al. dataset be scaled and centered based on the mean and variance of the Ramlall et al. dataset, which is a modification to other machine learning methods of applying the centering/scaling transformation derived from the training dataset.

TABLE 5
Evaluation of 2-Gene Sets on Ramlall et al. Dataset. AUC values
are provided, and Numbers in parentheses are standard deviations.

| | 5-fold Cross-Validation, 10000 rounds | | | Trained on 70% Training Sample | |
|---|---|---|---|---|---|
| | 70% Training | 30% Validation | Ramlall et | 30% Validation | Ramlall et |
| | Set | Set | al. Dataset | Set | al. Dataset |
| Gene Set | (n = 222) | (n = 96) | (n = 553) | (n = 96) | (n = 553) |
| IFI6, PTAFR | 0.960 (0.004) | 0.880 (0.017) | 0.895 (0.004) | 0.858 | 0.890 |
| IFI6, GBP5 | 0.955 (0.004) | 0.910 (0.014) | 0.902 (0.004) | 0.868 | 0.911 |
| IFI6, GRINA | 0.954 (0.004) | 0.916 (0.014) | 0.875 (0.004) | 0.896 | 0.888 |
| IFI44, TPM4 | 0.946 (0.004) | 0.837 (0.020) | 0.880 (0.004) | 0.772 | 0.877 |
| IFI44, BAZ1A | 0.944 (0.004) | 0.889 (0.017) | 0.893 (0.003) | 0.785 | 0.885 |
| IFI44, VCAN | 0.944 (0.004) | 0.841 (0.018) | 0.897 (0.003) | 0.806 | 0.883 |
| IFI44L, GBP5 | 0.952 (0.004) | 0.865 (0.017) | 0.910 (0.004) | 0.851 | 0.917 |
| IFI44L, BAZ1A | 0.947 (0.004) | 0.854 (0.019) | 0.879 (0.004) | 0.824 | 0.882 |
| IFI44L, SH3BP2 | 0.946 (0.004) | 0.857 (0.016) | 0.884 (0.003) | 0.794 | 0.859 |

### D. Gene Sets for Distinguishing SARS-COV-2+ and SARS-COV-2− Samples by PCR and LAMP Assay

The greedy selection algorithm was also applied to a version of the combined dataset that was not centered or scaled. Because the centering and scaling process minimizes the effects of relative expression differences between genes, skipping these steps could allow the selection algorithm to pick higher expressed genes, which might be advantageous for purposes of PCR. Results of this approach, including evaluation of the gene sets on the 70% training set and the 30% testing set, are shown in Table 6. Many of the same first and second genes were identified, though some of the top pairings are different. For example, PTAFR was identified as a second gene, but in combination with IFI44 rather than IFI6. In addition, omitting scaling and centering seemed to negatively impact the ability of the models to generalize to the 30% validation set, as the AUC scores in the final column are generally lower than those in the equivalent column in Table 2.

TABLE 6
Best Performing 2-Gene Sets on Filtered Combined Dataset (no scaling/centering).
Numbers in parentheses are standard deviations.

| | 70% Training Set | 30% Validation Set | 30% Validation Set |
|---|---|---|---|
| | (n = 222, 5-fold | (n = 96, 5-fold | (n = 96, trained on |
| Gene Set | CV, 10000 rounds) | CV, 10000 rounds) | 70% training sample) |
| IFI6, GBP5 | 0.951 (0.006) | 0.889 (0.025) | 0.887 |
| IFI6, GLUL | 0.949 (0.008) | 0.845 (0.021) | 0.859 |
| IFI6, GRINA | 0.950 (0.009) | 0.887 (0.017) | 0.907 |
| IFI44L, GBP2 | 0.928 (0.011) | 0.813 (0.022) | 0.840 |
| IFI44L, TPM4 | 0.928 (0.007) | 0.861 (0.022) | 0.851 |
| IFI44L, SH3BP2 | 0.925 (0.008) | 0.812 (0.023) | 0.806 |
| IFI44, PSTPIP2 | 0.948 (0.004) | 0.804 (0.022) | 0.770 |
| IFI44, PTAFR | 0.946 (0.006) | 0.874 (0.015) | 0.808 |
| IFI44, BAZ1A | 0.945 (0.005) | 0.849 (0.022) | 0.785 |

We further optimized classifier gene selection for performance on a PCR platform, where ensuring target genes have maximal differences in gene expression fold changes versus each other is much more critical than for mNGS. As an alternative approach to identifying genes that might be useful in distinguishing SARS-COV-2+ and SARS-COV-2− samples by PCR, we employed an intersection approach by combining the greedy selection algorithm with Differential Expression (DE) analysis (see Section VII.B.2). The analysis was performed separately on the Mick et al. dataset and the Ramlall et al. dataset, thus producing two independent lists. The top 20 genes from each dataset are shown in Table 7. The top five genes on the combined dataset (IFI6, IFI27, IFI44, USP18, and IFI44L) are also present in the top 20 list for the Ramlall et al. dataset (at positions 1, 2, 7, 3, and 16).

TABLE 7
Ranked list of first genes from Combined
dataset and Ramlall et al. dataset

| | |
|---|---|
| Combined Dataset | Ramlall et al. Dataset |

| | Gene | AUC Score | Gene | AUC Score |
|---|---|---|---|---|
| 1 | IFI6 | 0.882246 | IFI6 | 0.87246 |
| 2 | IFI27 | 0.865209 | IFI27 | 0.862661 |
| 3 | IFI44 | 0.859642 | USP18 | 0.855067 |
| 4 | USP18 | 0.84969 | BST2 | 0.850738 |
| 5 | IFI44L | 0.847157 | DDX60 | 0.847792 |
| 6 | HERC6 | 0.846878 | CMPK2 | 0.846746 |
| 7 | RTP4 | 0.845228 | IFI44 | 0.845948 |
| 8 | IFIT1 | 0.841351 | OAS2 | 0.845336 |
| 9 | BST2 | 0.831223 | SHFL | 0.845091 |
| 10 | LY6E | 0.829293 | MX1 | 0.844864 |
| 11 | LGALS3BP | 0.827874 | HERC6 | 0.844141 |
| 12 | ISG15 | 0.827155 | IFIT1 | 0.841163 |
| 13 | CMPK2 | 0.823177 | XAF1 | 0.840085 |
| 14 | RSAD2 | 0.812829 | SPATS2L | 0.839302 |
| 15 | OAS2 | 0.811829 | PARP12 | 0.837943 |
| 16 | EPSTI1 | 0.810357 | IFI44L | 0.836657 |
| 17 | MX1 | 0.802794 | EIF2AK2 | 0.830274 |
| 18 | IFIT5 | 0.802423 | OAS3 | 0.821089 |
| 19 | DDX58 | 0.799187 | DHX58 | 0.818051 |
| 20 | IFITM3 | 0.796163 | NRIR | 0.815679 |

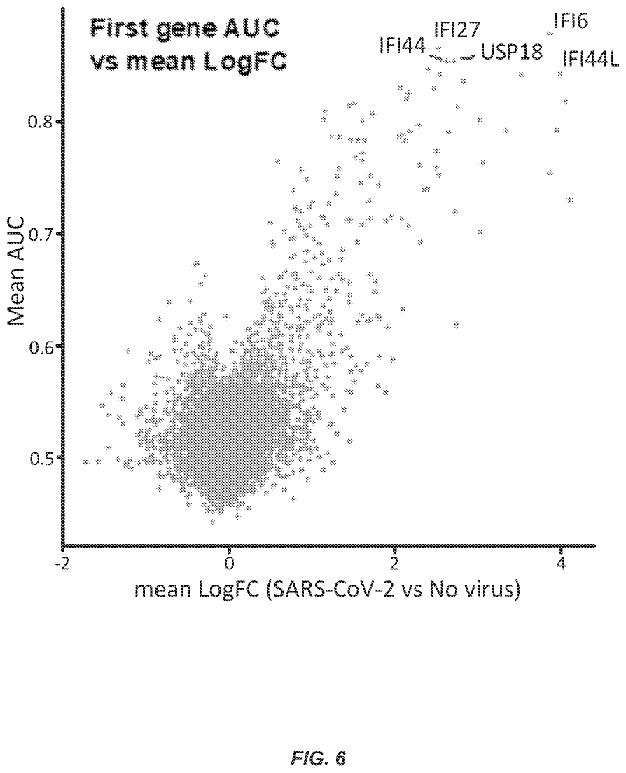

FIG. 6 shows mean AUC (averaged over the Mick et al. dataset and the Ramlall et al. dataset) of single genes plotted as a function of mean log fold change illustrating performance of single genes to differentiate COVID-19 from non-viral acute respiratory illnesses when incorporating fold-change.

Candidate genes that performed best, i.e., with the maximal AUC and fold changes between comparator groups were then selected and are shown in Table 8.

TABLE 8
Best performing single genes with respect to no virus comparator groups.

| | | | | UCSF COVID | Ramlall et al. |
|---|---|---|---|---|---|
| | | Ramlall | | vs. no | COVID vs. no |
| Gene | UCSF AUC | et al. AUC | Mean AUC | virus logFC | virus logFC |
| IFI6 | 0.882 (#1) | 0.872 (#1) | 0.877 (#1) | 3.89 | 3.82 |
| IFI27 | 0.865 (#2) | 0.862 (#2) | 0.864 (#2) | 2.74 | 2.3 |
| IFI44 | 0.86 (#3) | 0.845 (#7) | 0.852 (#3) | 2.57 | 2.65 |
| USP18 | 0.85 (#4) | 0.855 (#3) | 0.852 (#4) | 2.54 | 2.86 |
| IFI44L | 0.847 (#5) | 0.836 (#16) | 0.842 (#6) | 3.6 | 4.37 |

A second round of the gene selection algorithm was then performed using each of these top 5 best performing genes as the starting point in order to generate ranked lists of second genes. The role of the second gene is to primarily serve to distinguish COVID-19 from other types of viral respiratory illnesses.

In total, 10 second gene lists were generated (5 genes×2 datasets), and the top 20 genes from each are shown in Tables 9 and 10.

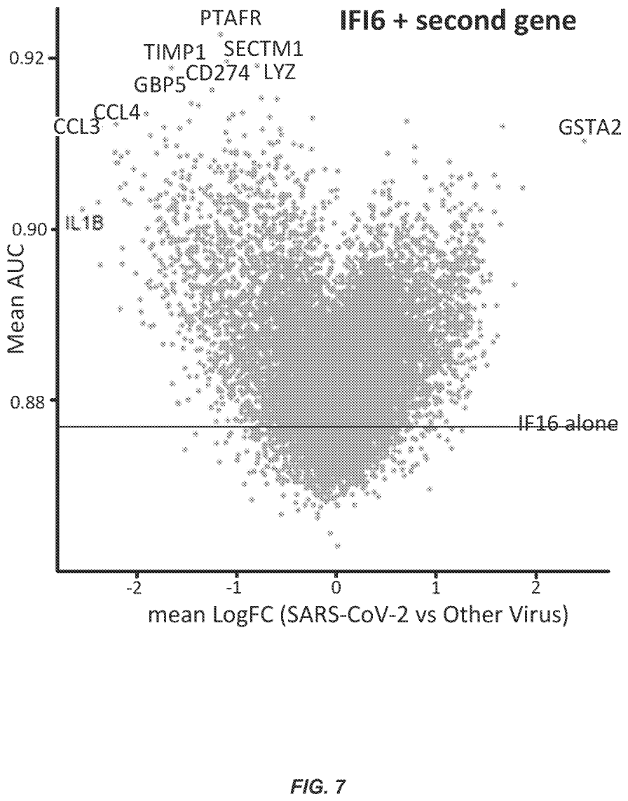

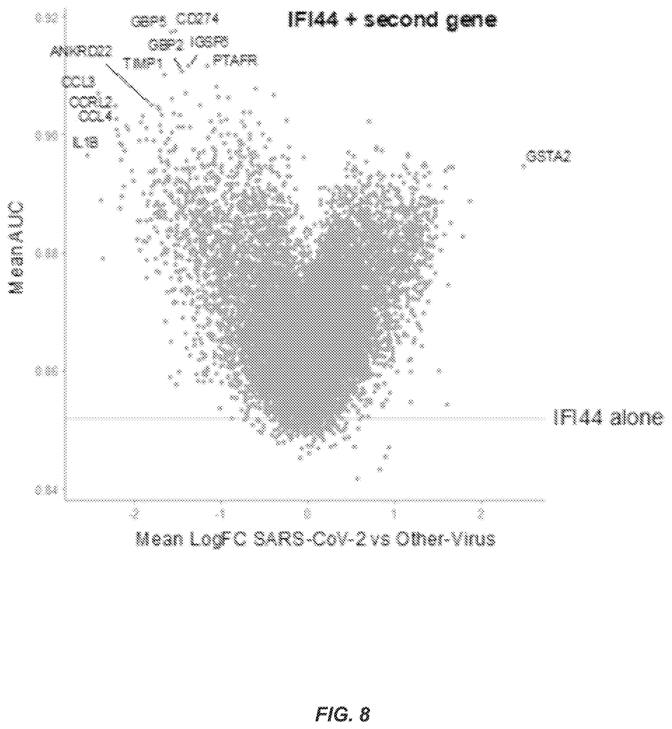

FIG. 7 shows mean AUC (averaged over the Mick et al. dataset and the Ramlall et al. dataset) of second genes paired with IFI6 plotted as a function of mean log fold change illustrating performance of second genes paired with IFI6 to differentiate COVID-19 from other virus when incorporating fold-change.

FIG. 8 shows mean AUC (averaged over the Mick et al. dataset and the Ramlall et al. dataset) of second genes paired with IFI44 plotted as a function of mean log fold change illustrating performance of second genes paired with IFI44 to differentiate COVID-19 from other virus when incorporating fold-change.

TABLE 9
Ranked list of second genes from Combined dataset

| | Paired with IFI6 | | Paired with IFI27 | | Paried with IFI44 | | Paired with USP18 | | Paired with IFI44L | |
|---|---|---|---|---|---|---|---|---|---|---|
| | Gene | AUC Score | Gene | AUC Score | Gene | AUC Score | Gene | AUC Score | Gene | AUC Score |
| 1 | GRINA | 0.952588 | ADD3 | 0.930571 | TIMP1 | 0.926706 | GBP5 | 0.904025 | TPM4 | 0.916574 |
| 2 | PTAFR | 0.949684 | BANK1 | 0.930174 | PTAFR | 0.926392 | TPM4 | 0.900008 | SERPINB | 0.915654 |
| 3 | GSTA2 | 0.945616 | CSTB | 0.926548 | TPM4 | 0.925932 | GBP1 | 0.899984 | ATP6V1 | 0.915048 |
| 4 | SNX10 | 0.943864 | SETD2 | 0.925016 | IRAK3 | 0.924115 | SERPINB9 | 0.898468 | B4GALT | 0.903064 |
| 5 | TIMP1 | 0.943004 | ITPKB | 0.924266 | CCRL2 | 0.922985 | SNRPN | 0.898071 | MAN1A | 0.901965 |
| 6 | CXCL16 | 0.940859 | RHEX | 0.924241 | BAZ1A | 0.922862 | ADH1C | 0.897801 | IL1RN | 0.900544 |
| 7 | FLOT1 | 0.940611 | CCR2 | 0.924147 | IGSF6 | 0.922325 | PKIG | 0.897119 | TPD52L2 | 0.899801 |
| 8 | CCL4 | 0.940391 | TGFBI | 0.924117 | CASP5 | 0.922229 | PSMD10 | 0.895698 | IRAK3 | 0.899506 |
| 9 | FGR | 0.940283 | CD22 | 0.923571 | ZNF267 | 0.921824 | DDX58 | 0.895563 | GBP2 | 0.899156 |
| 10 | SERPINB9 | 0.940279 | CBFA2 | 0.923567 | VCAN | 0.920847 | SLAMF7 | 0.895515 | TCEAL3 | 0.898237 |
| 11 | CEBPB | 0.939574 | HMOX | 0.92333 | SERPINB9 | 0.920744 | STX11 | 0.895375 | MRPL40 | 0.898233 |
| 12 | GSTA1 | 0.939059 | ATAD5 | 0.92316 | GBP2 | 0.920631 | NGRN | 0.895151 | ARHGEF | 0.898072 |
| 13 | CD68 | 0.938979 | MDM | 0.923075 | PADI2 | 0.920619 | STAT1 | 0.894311 | NDUFA5 | 0.897618 |
| 14 | SIRPA | 0.93892 | SLAMF | 0.922848 | CD93 | 0.920015 | TOMM34 | 0.894234 | SIRPB2 | 0.897108 |
| 15 | DENND5A | 0.938376 | SLAMF | 0.922749 | DENNDSA | 0.919944 | DALRD3 | 0.893785 | SDHAF1 | 0.896977 |
| 16 | XRN1 | 0.93834 | PADI2 | 0.922587 | DSC2 | 0.919631 | GBP4 | 0.893253 | SPCS1 | 0.89695 |
| 17 | SECTM1 | 0.93798 | PNOC | 0.922423 | PGK1 | 0.919489 | RIPK2 | 0.892729 | FTH1 | 0.896647 |
| 18 | CD93 | 0.937916 | STEAP | 0.922364 | RALGDS | 0.919105 | PTAFR | 0.892572 | SPN | 0.896389 |
| 19 | NINJ1 | 0.937863 | CD8A | 0.922186 | GBP5 | 0.918948 | BRD3OS | 0.892553 | MPP1 | 0.895618 |
| 20 | SLAMF7 | 0.937841 | IL24 | 0.921752 | CD274 | 0.918261 | PITPNC1 | 0.891836 | EGR2 | 0.895525 |

TABLE 10
Ranked list of second genes from Ramlall et al. dataset

| | Paired with IFI6 | | Paired with IFI27 | | Paried with IF44 | | Paired with USP18 | | Paired with IFI44L | |
|---|---|---|---|---|---|---|---|---|---|---|
| | Gene | AUC Score | Gene | AUC Score | Gene | AUC Score | Gene | AUC Score | Gene | AUC Score |
| 1 | HM13 | 0.910531 | RPL27 | 0.907045 | CD274 | 0.916815 | GBP4 | 0.89955 | CD274 | 0.914712 |
| 2 | AC127526.5 | 0.907349 | ATL3 | 0.906264 | GBP5 | 0.915679 | LRRC8C | 0.896661 | GBP5 | 0.910593 |
| 3 | IDO1 | 0.906523 | PDIA3 | 0.90608 | CYBB | 0.910629 | BATF2 | 0.896416 | WEE1 | 0.90811 |
| 4 | CYBB | 0.906061 | IDH1 | 0.905784 | ANKRD22 | 0.90664 | LILRB1 | 0.894505 | CAMSAP1 | 0.907882 |
| 5 | LYZ | 0.905722 | DYNC112 | 0.905576 | C2CD3 | 0.904999 | FAM20A | 0.894072 | NEDD4L | 0.904069 |
| 6 | PFN1 | 0.90562 | CHD2 | 0.904595 | CAMSAP1 | 0.904917 | IDO1 | 0.892813 | GCNT2 | 0.903385 |
| 7 | CD274 | 0.904022 | XRCC6 | 0.904563 | ACSL4 | 0.904329 | WARS1 | 0.89233 | SECTM1 | 0.902374 |
| 8 | HPSE | 0.902025 | UGP2 | 0.904439 | CTSS | 0.903326 | ARHGAP31 | 0.891733 | CYBB | 0.901164 |
| 9 | AC092868.3 | 0.901981 | RPN2 | 0.904387 | ZBTB10 | 0.903088 | GBP1P1 | 0.891341 | P2RY14 | 0.900844 |
| 10 | TOP1 | 0.901513 | AC009716. | 0.904039 | WEE1 | 0.902828 | HAVCR2 | 0.891122 | IFFO2 | 0.900831 |
| 11 | GCNT2 | 0.901342 | KLHL15 | 0.904014 | HM13 | 0.902807 | TIMP1 | 0.891103 | SLC25A25 | 0.900406 |
| 12 | ACOD1 | 0.9013 | KDM2A | 0.903965 | GBP2 | 0.90275 | PFN1 | 0.89084 | LYZ | 0.900351 |
| 13 | SECTM1 | 0.901158 | HADHB | 0.90386 | FFO2 | 0.902733 | LAIR1 | 0.89083 | USP2 | 0.898992 |
| 14 | AC093591.3 | 0.90089 | TLE4 | 0.90379 | LTA4H | 0.902147 | CXCL9 | 0.890679 | ALKBH5 | 0.898581 |
| 15 | CALHM6 | 0.900705 | CALR | 0.903437 | SECTM1 | 0.902081 | MTHFD2 | 0.890555 | CHKA | 0.898502 |
| 16 | C4BPA | 0.90024 | USO1 | 0.903072 | ASXL1 | 0.901743 | CYBB | 0.890072 | PTAFR | 0.89836 |
| 17 | P2RX7 | 0.900204 | LYPLA1 | 0.902876 | AIM2 | 0.901268 | NEDD4L | 0.889277 | FCGR1A | 0.898236 |
| 18 | GBP2 | 0.900159 | RPS29 | 0.902743 | USP54 | 0.900954 | IL18BP | 0.889162 | AC127526. | 0.898149 |
| 19 | GBP5 | 0.899929 | WDR26 | 0.902497 | TBC1D8 | 0.900637 | VAMP5 | 0.888715 | TUBB2A | 0.898059 |
| 20 | NR1D2 | 0.899754 | SEC31A | 0.902203 | FCGR1A | 0.900345 | MPZL1 | 0.888643 | NHSL1 | 0.897992 |

We then identified the optimal second gene based on AUC and maximal fold change differences between SARS-COV-2 and Other Virus classifier groups which resulted in a set of 2 gene combinations (Table 11). Table 11 shows host gene pairs optimized for PCR or LAMP based diagnostic platforms with best performance for COVID-19 diagnosis based on a combination of AUC from mNGS and fold change difference in expression. In some approaches, the gene expression level of two gene markers of a two-gene set from Table 11 can be measured along with detection of a normalization gene (e.g., RNaseP) and a viral gene marker (e.g., viral E gene target) in a PCR or LAMP assay (Table 11).

TABLE 11
Host gene pairs identified using the intersection approach.

| First Gene | Second gene |
|---|---|
| IFI6 | GSTA2 |
| IFI6 | GBP5 |
| IFI6 | CCL3 |
| IFI44L | GSTA2 |
| IFI44L | GBP5 |
| IFI44L | CCL3 |
| IFI27 | GSTA2 |
| IFI27 | GBP5 |
| IFI27 | CCL3 |

### V. Gene Signatures

As described above, we observed changes in the transcriptome of subjects diagnosed with COVID-19 and identified genes whose expression levels were found to be altered by COVID-19. We show that these genes perform well in distinguishing COVID-19 samples from other ARI samples. The identified genes may be combined and used in gene panels as suitable for a given diagnostic method. This section summarizes the identified genes and provides examples of gene panels, including two-gene panels.

Section V(A), below, describes host genes that can be monitored (e.g., using a PCR or LAMP assay) to identify subjects with COVID-19. A host gene set includes a pair of host genes that together can be used for determining Covid-19 status and other uses. Each pair comprises a first gene and a second gene. In some approaches, the pair are the only host genes evaluated in an assay. In another approach, other host genes are assayed and the pair are used in combination with the other host genes. In some cases the other host gene is an additional first gene, described below. In some cases the other host gene is an additional second gene, described below. Optionally, detection of expression from host gene sets can be paired with a host control gene (e.g., RNAseP control gene) and a single viral (e.g., E gene) target in a rapid PCR or LAMP assay. In some embodiments the host gene set is measured along with more than one viral genes.

Section V(B) describes sets of host genes that can be monitored (e.g., using a PCR or LAMP assay) to identify subjects with COVID-19. These host sets were selected based on identifying genes with the greatest combinations of mean AUC and mean fold change for both the COVID-19 vs No-Virus (1st gene) and SARS-CoV-2 vs Other-Virus (2nd gene) comparisons. This resulted in an optimized set of host gene pairs for assessing subjects describes host genes that can be monitored (e.g., using a PCR or LAMP assay) to identify subjects with COVID-19. A host gene set includes a pair of host genes that together can be used for determining Covid-19 status and other uses. Each pair comprises a first gene and a second gene. In some approaches, the pair are the only host genes evaluated in an assay. In another approach, other host genes are assayed and the pair are used in combination with the other host genes. In some cases the other host gene is an additional first gene, described below. In some cases the other host gene is an additional second gene, described below. Optionally, detection of expression from host gene sets can be paired with a host control gene (e.g., RNAseP control gene) and a single viral (e.g., E gene) target in a rapid PCR or LAMP assay. In some embodiments the host gene set is measured along with more than one viral genes.

Section V(C) describes detection of viral markers in combination with a host gene pair.

Section V(D) describes use of a use of normalization marker.

### A. Host Gene Sets—Group 1

The genes identified through the analyses described in Section IV and as summarized in Tables 2-9 are listed in Table 12A-C. Thus, provided herein are the genes shown in Table 12A-C and their diagnostic uses for assessing COVID-19 disease and/or SARS-COV-2 infection. In some embodiments, one or more genes disclosed herein have a differential expression induced by SARS-COV-2. In some embodiments of the compositions and methods described herein, a plurality of the genes listed in Table 12A-C can be used to identify and diagnose COVID-19 in a subject. Sequence identifiers are provided, but it will be understood that gene markers include variants (e.g., polymorphic variants, etc.) of the identified genes. Tables 12A and 12B provide annotations for first genes and second genes, including Entrez Gene IDs and HGNC IDs.

It will be understood that a reference to “Table 12” can refer to one of more of Tables 12A, 12B, and 12C, as will be clear from context. Likewise it will be understood that a reference to “Table 13” can refer to one of more of Table 13A, 13B, 13C and 13D, as will be clear from context.

Any combination of a first gene (Table 12A) and a second gene (Table 12B) may be used in assays of the invention. Table 12C lists exemplary two-gene sets.

TABLE 12A
FIRST GENES AND ANNOTATIONS

| Gene Symbol | Gene Name | Synonym | Entrez Gene ID | HGNC ID |
|---|---|---|---|---|
| IFI6 | interferon alpha inducible | 6-16, FAM14C, | 2537 | 4054 |
| | protein 6 | G1P3, IFI-6-16, | | |
| | | IFI616 | | |
| IFI27 | interferon alpha inducible | FAM14D, ISG12, | 3429 | 5397 |
| | protein 27 | ISG12A, P27 | | |
| IFI44 | interferon induced protein | MTAP44, TLDC5, | 10561 | 16938 |
| | 44 | p44 | | |
| IFI44L | interferon induced protein | C1orf29, GS3686 | 10964 | 17817 |
| | 44 like | | | |

TABLE 12B
SECOND GENES AND ANNOTATIONS

| Gene Symbol | Gene Name | Synonym | Entrez Gene ID | HGNC ID |
|---|---|---|---|---|
| ATP13A4 | ATPase 13A4 | | 84239 | 25422 |
| ATP5F1C | ATP synthase F1 subunit | ATP5C, ATP5C1, | 509 | 833 |
| | gamma | ATPSCL1 | | |
| BAZ1A | bromodomain adjacent to | ACF1, WALp1, | 11177 | 960 |
| | zinc finger domain 1A | WCRF180, hACF1 | | |
| CCL3 | C-C motif chemokine ligand | G0S19-1, | 6348 | 10627 |
| | 3 | LD78ALPHA, MIP-1- | | |
| | | alpha, MIP1A, | | |
| | | SCYA3 | | |
| GBP5 | guanylate binding protein 5 | GBP-5 | 115362 | 19895 |
| GLUL | glutamate-ammonia ligase | GLNS, GS, PIG43, | 2752 | 4341 |
| | | PIG59 | | |
| GRIN2C | glutamate ionotropic | GluN2C, NMDAR2C, | 2905 | 4587 |
| | receptor NMDA type | NR2C | | |
| | subunit 2C | | | |
| GRINA | glutamate ionotropic | HNRGW, LFG1, | 2907 | 4589 |
| | receptor NMDA type | NMDARA1, TMBIM3 | | |
| | subunit associated protein 1 | | | |
| GSTA2 | glutathione S-transferase | GST2, GSTA2-2, | 2939 | 4627 |
| | alpha 2 | GTA2, GTH2 | | |
| MMADHC | metabolism of cobalamin | C2orf25, CL25022, | 27249 | 25221 |
| | associated D | cblD | | |
| OR2C3 | olfactory receptor family 2 | OR2C4, OR2C5P, | 81472 | 15005 |
| | subfamily C member 3 | OST742 | | |
| OTUD7A | OTU deubiquitinase 7A | C15orf16, | 161725 | 20718 |
| | | C16ORF15, | | |
| | | CEZANNE2, OTUD7 | | |
| PBDC1 | polysaccharide biosynthesis | CXorf26 | 51260 | 28790 |
| | domain containing 1 | | | |
| PDGFRB | platelet derived growth | CD140B, IBGC4, | 5159 | 8804 |
| | factor receptor beta | IMF1, JTK12, KOGS | | |
| PLK4 | polo like kinase 4 | MCCRP2, SAK, | 10733 | 11397 |
| | | STK18 | | |
| POP1 | POP1 homolog, | ANXD2 | 10940 | 30129 |
| | ribonuclease P/MRP subunit | | | |
| POSTN | periostin | OSF-2, OSF2, | 10631 | 16953 |
| | | PDLPOSTN, PN | | |
| PSTPIP2 | proline-serine-threonine | MAYP | 9050 | 9581 |
| | phosphatase interacting | | | |
| | protein 2 | | | |
| PTAFR | platelet activating factor | PAFR | 5724 | 9582 |
| | receptor | | | |
| SH3BP2 | SH3 domain binding protein | 3BP-2, 3BP2, CRBM, | 6452 | 10825 |
| | 2 | CRPM, RES4-23 | | |
| TPM4 | tropomyosin 4 | HEL-S-108 | 7171 | 12013 |
| USP18 | ubiquitin specific peptidase | ISG43, PTORCH2, | 11274 | 12616 |
| | 18 | UBP43 | | |
| VCAN | versican | CSPG2, ERVR, GHAP, | 1462 | 2464 |
| | | PG-M, WGN | | |
| WASF1 | WASP family member 1 | NEDALVS, SCAR1, | 8936 | 12732 |
| | | WAVE, WAVE1 | | |
| WDR74 | WD repeat domain 74 | Nsa1 | 54663 | 25529 |

TABLE 12C
EXEMPLARY TWO-GENE SETS

| First | Second | First | Second | First | Second | First | Second |
|---|---|---|---|---|---|---|---|
| Gene | Gene | Gene | Gene | Gene | Gene | Gene | Gene |
| IFI6 | ATP13A4 | IFI44 | ATP13A4 | IFI44L | ATP13A4 | IFI27 | ATP13A4 |
| IFI6 | ATP5F1C | IFI44 | ATP5F1C | IFI44L | ATP5F1C | IFI27 | ATP5F1C |
| IFI6 | BAZ1A | IFI44 | BAZ1A | IFI44L | BAZ1A | IFI27 | BAZ1A |
| IFI6 | CCL3 | IFI44 | CCL3 | IFI44L | CCL3 | IFI27 | CCL3 |
| IFI6 | GBP5 | IFI44 | GBP5 | IFI44L | GBP5 | IFI27 | GBP5 |
| IFI6 | GLUL | IFI44 | GLUL | IFI44L | GLUL | IFI27 | GLUL |
| IFI6 | GRIN2C | IFI44 | GRIN2C | IFI44L | GRIN2C | IFI27 | GRIN2C |
| IFI6 | GRINA | IFI44 | GRINA | IFI44L | GRINA | IFI27 | GRINA |
| IFI6 | GSTA2 | IFI44 | GSTA2 | IFI44L | GSTA2 | IFI27 | GSTA2 |
| IFI6 | MMADHC | IFI44 | MMADHC | IFI44L | MMADHC | IFI27 | MMADHC |
| IFI6 | OR2C3 | IFI44 | OR2C3 | IFI44L | OR2C3 | IFI27 | OR2C3 |
| IFI6 | OTUD7A | IFI44 | OTUD7A | IFI44L | OTUD7A | IFI27 | OTUD7A |
| IFI6 | PBDC1 | IFI44 | PBDC1 | IFI44L | PBDC1 | IFI27 | PBDC1 |
| IFI6 | PDGFRB | IFI44 | PDGFRB | IFI44L | PDGFRB | IFI27 | PDGFRB |
| IFI6 | PLK4 | IFI44 | PLK4 | IFI44L | PLK4 | IFI27 | PLK4 |
| IFI6 | POP1 | IFI44 | POP1 | IFI44L | POP1 | IFI27 | POP1 |
| IFI6 | POSTN | IFI44 | POSTN | IFI44L | POSTN | IFI27 | POSTN |
| IFI6 | PSTPIP2 | IFI44 | PSTPIP2 | IFI44L | PSTPIP2 | IFI27 | PSTPIP2 |
| IFI6 | PTAFR | IFI44 | PTAFR | IFI44L | PTAFR | IFI27 | PTAFR |
| IFI6 | SH3BP2 | IFI44 | SH3BP2 | IFI44L | SH3BP2 | IFI27 | SH3BP2 |
| IFI6 | TPM4 | IFI44 | TPM4 | IFI44L | TPM4 | IFI27 | TPM4 |
| IFI6 | USP18 | IFI44 | USP18 | IFI44L | USP18 | IFI27 | USP18 |
| IFI6 | VCAN | IFI44 | VCAN | IFI44L | VCAN | IFI27 | VCAN |
| IFI6 | WASF1 | IFI44 | WASF1 | IFI44L | WASF1 | IFI27 | WASF1 |
| IFI6 | WDR74 | IFI44 | WDR74 | IFI44L | WDR74 | IFI27 | WDR74 |

As described herein, the compositions and methods may use a host gene panel comprising one or more genes selected form the group of genes listed in Table 12. In some embodiments, the expression levels of one or more of these genes may change (e.g., increase or decrease) as induced by COVID-19 disease and/or SARS-COV-2 infection. In some embodiments, the expression levels of one or more of these genes may increase or decrease as induced by COVID-19 disease and/or SARS-COV-2 infection. In some embodiments, the expression levels of one or more of these genes may be increased or decreased by COVID-19 disease as compared to a non-viral ARI. In some embodiments, the expression levels of one or more of these genes may be increased or decreased by COVID-19 disease as compared to an ARI caused by another virus.

As non-limiting examples, the genes may have polynucleotide sequences as specified in Table 12. In some embodiments, the expression level of a variant of a gene as listed in Table 12 may be measured. For example, the gene may be a polymorphic variant of a gene as shown in Table 12. In some embodiments, the gene may comprise a polymorphism (e.g., a single nucleotide polymorphism). In some embodiments, the gene may have a sequence that is at least 85% identical to a sequence listed in Table 12, such as at least 90%, 91%, 92%, 93%, 94%, 95%, 96%, 97%, 98% or at least 99% identical to a sequence listed in Table 12.

In some embodiments, the expression level of one single gene is used for the diagnosis of COVID-19. In some embodiments, the expression level of at least one gene selected from the group consisting of genes listed in Table 12 is measured. In certain embodiments, a panel of two or more gene markers listed in Table 12 is used for the diagnosis of COVID-19. The gene panel may comprise any suitable number of genes selected from the genes listed in Table 12. Gene panels may comprise between 2 to 29 gene markers, inclusive, including for example 3, 4, 5, 6, 7, 8, 9, 10, 11, 12, 13, 14, 15, 16, 17, 18, 19, 20, 21, 22, 23, 24, 25, 26, 27, 28, or 29 gene markers selected from the gene markers as listed in Table 12. In certain embodiments, the invention includes a gene marker panel comprising at least 2, at least 3, at least 4, at least 5, or at least 6 or more gene markers selected from the gene markers as listed in Table 12. For example, a method disclosed herein, may comprise measuring the expression level of two or more, three or more, four or more, five or more, six or more, or seven or more genes selected from the group consisting of genes listed in Table 12. In some embodiments, a gene marker panel for the diagnosis of COVID-19 comprises no more than two, no more than three, or no more than four gene markers selected from the group of gene markers listed in Table 12.

The expression level of any combination of 2, 3, 4, 5, 6 or more genes as shown in Table 12A-B may be measured in the biological sample. As described in Section IV, we identified two-gene sets using a variety of approaches (see Tables 2, 3, 5, and 6). Thus, in certain embodiments, a method as disclosed herein may comprise measuring, in the biological sample, the expression level of two genes selected from the group of two-gene sets listed in Table 12C.

In some approaches, expression levels of the genes shown in Table 12A-B (or of the two genes shown as pairwise combination in Table 12C) are measured using amplification methods (e.g., PCR-based methods or LAMP assays, described in sections VIII.A and VIII.B, below). As described in section IV.D above, we identified genes and gene sets that may be particularly well suited as diagnostic gene markers when using PCR or LAMP platforms. Accordingly, a method as disclosed herein may comprise measuring the expression level of one or more genes selected from the group consisting of IFI6, IFI27, IFI44, USP18, and IFI44L, where measuring the expression level comprises performing a PCR (e.g., RT-PCR) or LAMP assay. In some approaches, a method as disclosed herein may comprise measuring the expression level of two genes selected from the group of two-gene sets consisting of IFI6 and GLUL, IFI6 and GRINA, IFI6 and GSTA2, IFI6 and GBP5, IFI6 and CCL3, IFI44L and GSTA2, IFI44L and GBP5, IFI44L and GBP2, IFI44L and TPM4, IFI44L and SH3BP2, IFI44L and CCL3, IFI27 and GSTA2, IFI27 and GBP5, IFI27 and CCL3, IFI44 and PSTPIP2, IFI44 and PTAFR, and IFI44 and BAZ1A, where measuring the expression level comprises performing a PCR (e.g., RT-PCR) or LAMP assay. In some approaches, the expression level of the first human gene (e.g., IFI6, IFI27, or IFI44) is used to determine the presence of a virus infection (irrespective of virus type) and the expression level of the second human gene (e.g., GSTA2, GLUL, or GBP5) is used to determine the presence of a SARS-COV-2 infection.

### 8. Host Gene Sets—Group 2

A list of candidate genes for PCR or LAMP assay were selected based on identifying genes with the greatest combinations of mean AUC and mean fold change for both the COVID-19 vs No-Virus (1st gene) and SARS-CoV-2 vs Other-Virus (2nd gene) comparisons. This resulted in a final set of 2 gene combinations. These combinations can be paired with detection of a control gene (e.g., RNAseP) and a single viral gene (e.g., E gene) target in an assay (e.g., a rapid PCR or LAMP assay. Thus, provided herein are the genes shown in Table 13A-D and their diagnostic uses for assessing COVID-19 disease and/or SARS-COV-2 infection. In some embodiments, one or more genes disclosed herein have a differential expression induced by SARS-COV-2. In some embodiments of the compositions and methods described herein, a plurality of the genes listed in Table 13A-C can be used to identify and diagnose COVID-19 in a subject. Sequence identifiers are provided, but it will be understood that gene markers include variants (e.g., polymorphic variants, etc.) of the identified genes. Tables 13D provides annotations for 4 second genes not listed in Table 12B, including Entrez Gene IDs and HGNC IDs.

Any combination of a first gene (Table 13A and 13B) and a second gene (Table 13B) may be used in assays of the invention. Table 13C lists exemplary two-gene sets.

Table 13D provides annotations for second genes not described in Table 12B. In some embodiments, the expression levels of one or more of these genes may change (e.g., increase or decrease) as induced by COVID-19 disease and/or SARS-COV-2 infection. In some embodiments, the expression levels of one or more of these genes may increase or decrease as induced by COVID-19 disease and/or SARS-COV-2 infection. In some embodiments, the expression levels of one or more of these genes may be increased or decreased by COVID-19 disease as compared to a non-viral ARI. In some embodiments, the expression levels of one or more of these genes may be increased or decreased by COVID-19 disease as compared to an ARI caused by another virus.

TABLE 13A
FIRST GENES

SARS-CoV-2 vs No-Virus

| 1^{st} Gene | Mean AUC | Mean log2FC |
|---|---|---|
| IFI6 | 0.877 | 3.859 |
| IFI27 | 0.864 | 2.520 |
| IFI44 | 0.853 | 2.612 |
| IFI44L | 0.842 | 3.986 |

TABLE 13B
SECOND GENES

| | IFI6 (1^{st} Gene) | | IFI44 (1^{st} Gene) | |
|---|---|---|---|---|
| | SARS-CoV-2 vs Other-Virus | | SARS-CoV-2 vs Other-Virus | |
| 2^{nd} Gene | Mean AUC | Mean log2FC | Mean AUC | Mean log2FC |
| ANKRD22 | 0.909 | −1.796 | 0.905 | −1.796 |
| CCL3 | 0.911 | −2.415 | 0.907 | −2.415 |
| CD274 | 0.917 | −1.523 | 0.918 | −1.523 |
| GBP5 | 0.916 | −1.581 | 0.917 | −1.581 |
| GSTA2 | 0.910 | 2.481 | 0.895 | 2.481 |
| PTAFR | 0.923 | −1.165 | 0.912 | −1.165 |
| SIGLEC10 | 0.905 | −1.547 | 0.904 | −1.547 |
| TIMP1 | 0.919 | −1.658 | 0.910 | −1.658 |

EXEMPLARY 13C-EXEMPLARY TWO-GENE SETS

| First | Second | First | Second | First | Second | First | Second |
|---|---|---|---|---|---|---|---|
| Gene | Gene | Gene | Gene | Gene | Gene | Gene | Gene |
| IFI6 | ANKRD22 | IFI44 | ANKRD22 | IFI27 | ANKRD22 | IFI44L | ANKRD22 |
| IFI6 | CCL3 | IFI44 | CCL3 | IFI27 | CCL3 | IFI44L | CCL3 |
| IFI6 | CD274 | IFI44 | CD274 | IFI27 | CD274 | IFI44L | CD274 |
| IFI6 | GBP5 | IFI44 | GBP5 | IFI27 | GBP5 | IFI44L | GBP5 |
| IFI6 | GSTA2 | IFI44 | GSTA2 | IFI27 | GSTA2 | IFI44L | GSTA2 |
| IFI6 | PTAFR | IFI44 | PTAFR | IFI27 | PTAFR | IFI44L | PTAFR |
| IFI6 | SIGLEC10 | IFI44 | SIGLEC10 | IFI27 | SIGLEC10 | IFI44L | SIGLEC10 |
| IFI6 | TIMP1 | IFI44 | TIMP1 | IFI27 | TIMP1 | IFI44L | TIMP1 |

TABLE 13D
SECOND GENES - ADDITIONAL ANNOTATIONS

| Gene Symbol | Gene Name | Synonym | Entrez Gene ID | HGNC ID |
|---|---|---|---|---|
| ANKRD22 | Ankyrin Repeat Domain 22 | ANKRD22 5 | 118932 | 28321 |
| CD274 | CD274 Molecule | PDCD1LG1 | 29126 | 17635 |
| SIGLEC10 | Sialic Acid Binding Ig Like | SLG2 | 89790 | 15620 |
| | Lectin 10 | | | |
| TIMP1 | Tissue Inhibitor Of | TIMP, CLGI | 7076 | 11820 |
| | Metalloproteinases 1 | | | |

As non-limiting examples, the genes may have polynucleotide sequences as specified in TABLE 13A-C. In some embodiments, the expression level of a variant of a gene as listed in TABLE 13A-C may be measured. For example, the gene may be a polymorphic variant of a gene as shown in TABLE 13A-C. In some embodiments, the gene may comprise a polymorphism (e.g., single nucleotide polymorphism). In some embodiments, the gene may have a sequence that is at least 85% identical to a sequence listed in TABLE 13A-C, such as at least 90%, 91%, 92%, 93%, 94%, 95%, 96%, 97%, 98% or at least 99% identical to a sequence listed in TABLE 13A-C.

In some embodiments, the expression level of one single gene is used for the diagnosis of COVID-19. In some embodiments, the expression level of at least one gene selected from the group consisting of genes listed in TABLE 13A-C is measured. In certain embodiments, a panel of two or more gene markers listed in TABLE 13 is used for the diagnosis of COVID-19. The gene panel may comprise any suitable number of genes selected from the genes listed in TABLE 13A-C. Gene panels may comprise between 2 to 12 gene markers, inclusive, including for example 3, 4, 5, 6, 7, 8, 9, 10, 11, or 12 gene markers selected from the gene markers as listed in TABLES 13A-B. In certain embodiments, the invention includes a gene marker panel comprising at least 2, at least 3, at least 4, at least 5, or at least 6 or more gene markers selected from the gene markers as listed in TABLE 13A-B. For example, a method disclosed herein, may comprise measuring the expression level of two or more, three or more, four or more, five or more, six or more, or seven or more genes selected from the group consisting of genes listed in TABLE 13A-C. In some embodiments, a gene marker panel for the diagnosis of COVID-19 comprises no more than two, no more than three, or no more than four gene markers selected from the group of gene markers listed in TABLE 13.

The expression level of any combination of 2, 3, 4, 5, 6 or more genes as shown in TABLE 13A-C may be measured in the biological sample. As described in Section IV, we identified two-gene sets using a variety of approaches (see Tables 2, 3, 5, and 6). Thus, in certain embodiments, a method as disclosed herein may comprise measuring, in the biological sample, the expression level of two genes selected from a first gene and a second gene listed in Table 13A-C.

In some approaches, expression levels of the genes shown in TABLE 13B or 13C are measured using amplification methods (e.g., PCR-based methods or LAMP assays, described in sections VIII.A and VIII.B, below). As described in section IV.D above, we identified genes and gene sets that may be particularly well suited as diagnostic gene markers when using PCR or LAMP platforms.

In other approaches, a method as disclosed herein may comprise measuring the expression level of one or more genes selected from the group consisting of IFI6, IFI27, IFI44, USP18, and IFI44L in conjunction with measuring, in the biological sample, a viral expression level of a SARS-COV-2 viral gene (e.g., a sequence encoding the E protein of SARS-COV-2). In some approaches, a method as disclosed herein may comprise measuring the expression level of two genes selected from the group of two-gene sets consisting of IFI6 and GSTA2, IFI6 and GBP5, IFI6 and CCL3, IFI44L and GSTA2, IFI44L and GBP5, IFI44L and CCL3, IFI27 and GSTA2, IFI27 and GBP5, IFI27 and CCL3 in conjunction with measuring, in the biological sample, a viral expression level of a SARS-COV-2 viral gene.

### C. Other Host Gene Pairs

In addition to host gene combinations from Tables 12 and 13, host gene pairs for use in the invention are provided in Tables 2, 3, 4 and 11, and. host gene single gene or 3-gene sets are shown in Table 5. For example, in one aspect the invention provides a method of determining whether or not a human subject is infected with SARS-COV-2, the method comprising: (a) receiving a biological sample collected from the subject, the biological sample including human RNA from cells of the subject; (b) measuring, in the biological sample, i) a first gene expression level of a first human gene selected from the group consisting of genes shown in any of Tables 2, 3, 4 and 11; ii) a second gene expression level of a second human gene selected from the group consisting of genes shown in Tables 2, 3, 4 and 11, wherein the second human gene is different from the first human gene; (c) detecting differences, if any, in the first gene expression level and the second gene expression level relative to reference expression levels characteristic of a human subject who is not infected with SARS-COV-2; (d) determining whether the subject is infected with SARS-COV-2 based on the differences, if any, determined in (c). In one approach, single host gene or 3-host gene sets are assayed in place of the first and second genes above.

### D. Combined Host-Viral Gene Sets

The host genes listed in Table 12 may be used as host gene markers for diagnosing COVID-19 either alone or in combination with a viral marker. In some approaches, expression levels of the genes shown in Table 12A-C or Table 13A-D (or of the two genes shown as pairwise combination) are measured in conjunction with detecting the presence and/or the quantity of a viral SARS-COV-2 RNA. As described in section IV.B, we identified host genes that perform particularly well in distinguishing between SARS-COV-2-positive and SARS-COV-2-negative samples in combination with a viral marker. Accordingly, aspects of the disclosure relate to a combined host-viral diagnostic method for assessing the presence of COVID-19 in a subject. In some embodiments, methods described herein further comprise measuring, in the biological sample, a viral expression level of a SARS-COV-2 viral gene. In some embodiments, the method includes measuring expression levels of viral RNA comprising a sequence that encodes for the Nucleocapsid (N), Envelope (E), or Spike (S) protein, the open reading frame 1ab (Orf1ab), and/or the SARS-COV-2 RNA-dependent RNA polymerase (RdRP) gene, which is located within Orf1ab. Any suitable sequence of the SARS-COV-2 RNA may be used as a viral marker in the combined host-viral diagnostic method. In some embodiments, the viral marker may be a sequence encoding the E protein of SARS-COV-2. In some embodiments, the viral marker may be a sequence encoding the N protein of SARS-COV-2. In some embodiments, two or more viral marker may be used. For example, a viral marker having a sequence encoding the N protein of SARS-COV-2 and a viral marker having a sequence encoding the E protein of SARS-COV-2 may be used in combination. Targets of the SARS-COV-2 RNA that may be suitable for the detection of SARS-COV-2 are known and described e.g., in Feng et al. (2020), “Molecular Diagnosis of COVID-19: Challenges and Research Needs,” Anal. Chem., 92, 10196-10209; and Kilic et al. (2020), “Molecular and immunological diagnostic tests of COVID-19: Current status and challenges, iScience, 23(8): 101406.

### E. Normalization Gene

The expression level of a normalization gene may be used to control for variations arising from RNA extraction, processing, and other variables. In some embodiments, the expression level of the first and second human genes in the biological sample is determined by normalizing a measured expression level with a control expression level of a human normalization gene. Normalization genes are generally so-called housekeeping genes, i.e., a gene that is required for the maintenance of essential functions of any cell type and that exhibits invariable expression levels. Housekeeping genes are typically constitutively expressed in all cells, at any development stage irrespective of pathophysiological state. Exemplary, housekeeping genes or normalization genes that may be used include, Ribonuclease P (RNaseP) gene, genes encoding α-actin, β-actin, 185 rRNA, 28S rRNA, albumin, and glyceraldehyde-3-phosphate dehydrogenase (GAPDH). Additional suitable housekeeping genes that can be used to carry out the methods described herein may be found in the HRT Atlas Database (www.housekeeping.unicamp.br; Hounkpe et al. (2020), “HRT Atlas v1.0 database: redefining human and mouse housekeeping genes and candidate reference transcripts by mining massive RNA-seq datasets,” Nucleic Acids Research: gkaa609). Methods for use of housekeeping gene expression to normalize gene expression levels are known, see e.g., Karge et al. (1998), “Quantification of mRNA by polymerase chain reaction (PCR),” Methods Mol. Biol. 110:43-61; Gilliland et al., “Competitive PCR for quantitation of mRNA,” In: Innis MA, ed. PCR protocols: a guide to methods and applications. San Diego: Academic Press, 1990: 60-9. In one embodiment, the normalization gene used is RNaseP. In some embodiments, the expression level of the first and second human genes in the biological sample is determined by normalizing a measured expression level with the expression level of RNaseP.

F. Genes and 3-Gene Sets for Use in Combination with Reads Per Million (rpm)

As shown in Table 4 above, we identified single genes and three-gene sets that perform well in combination with rpM. Accordingly, in some approaches, a method as disclosed herein may comprise measuring the expression level of one or more genes selected from the group consisting of single genes or three-gene sets (sets 1-8) shown in Table 14 in conjunction with measuring, in the biological sample, a viral expression level of a SARS-COV-2 viral gene (e.g., a gene encoding the E protein of SARS-COV-2).

TABLE 14
Exemplary host genes for combined host-viral diagnostic assays

| | |
|---|---|
| 1 | WDR74 |
| 2 | PDGFRB |
| 3 | WDR74, ATP13A4, OTUD7A |
| 4 | WDR74, PLK4, PDGFRB |
| 5 | WDR74, OR2C3, WASF1 |
| 6 | PDGFRB, PBDC1, POP1 |
| 7 | PDGFRB, POSTN, MMADHC |
| 8 | PDGFRB, ATP5F1C, GRIN2C |

In other approaches, a method as disclosed herein may comprise measuring the expression level of one or more genes selected from the group consisting of IFI6, IFI27, IFI44, USP18, and IFI44L in conjunction with measuring, in the biological sample, a viral expression level of a SARS-COV-2 viral gene (e.g., a sequence encoding the E protein of SARS-COV-2). In some approaches, a method as disclosed herein may comprise measuring the expression level of two genes selected from the group of two-gene sets consisting of IFI6 and GSTA2, IFI6 and GBP5, IFI6 and CCL3, IFI44L and GSTA2, IFI44L and GBP5, IFI44L and CCL3, IFI27 and GSTA2, IFI27 and GBP5, IFI27 and CCL3 in conjunction with measuring, in the biological sample, a viral expression level of a SARS-COV-2 viral gene.

### G. Detecting Viral Infection

The 2-gene classifiers to not only serve as a COVID-19 diagnostic, but also as a universal viral diagnostic, given that expression of the interferon-induced genes (IFI6, IFI27, IFI44, IFI44L) is induced non-specifically by a diversity of respiratory viral pathogens. In some approaches, the expression level of the first human gene (e.g., IFI6, IFI27, or IFI44) is used to determine the presence of a virus infection (irrespective of virus type) and the expression level of the second human gene (e.g., GSTA2, GLUL, or GBP5) is used to determine the presence of a SARS-COV-2 infection. Respiratory viral pathogens that can be detected include influenza virus, respiratory syncytial virus, parainfluenza viruses, metapneumovirus, rhinovirus, coronaviruses, adenoviruses, and bocaviruses.

### VI. Diagnostic and Prognostic Methods

The gene markers and trained machine learning methods described herein are useful for various medical applications including COVID-19 diagnosis and prognosis and determining treatment responsiveness. The methods provided herein may be used to provide predictive analytics using machine learning-based approaches to analyze acquired data from a subject to generate an output of diagnosis of the subject, i.e., whether the subject has COVID-19. For example, the methods provided herein may be used to generate the diagnosis of the subject having COVID-19. As described in section III, the host gene markers were identified based on cohorts consisting of subjects that were in their acute illness phase, that is when they were presenting symptoms but were not in need of intubation. Thus, the methods provided herein may be particularly useful for the diagnosis of COVID-19 in the early stages of the illness when the subjects present with one or more acute respiratory illness symptoms. In some embodiments, the subject is suffering from one or more symptoms of COVID-19 (such as fever, cough, fatigue, breathing difficulties, and loss of smell and taste). In some embodiments, the subject is suspected of having COVID-19. In some embodiments, the subject is suspected of having a SARS-COV-2 (SARS-COV-2) infection.

Accordingly, provided herein is a method of determining SARS-COV-2 (SARS-COV-2) infection in a human subject, the method comprising (a) receiving a biological sample collected from the subject, the biological sample including human RNA from cells of the subject and SARS-COV-2 viral RNA, if present, (b) measuring, in the biological sample, (i) a first gene expression level of a first human gene selected from the group consisting of genes shown in Table 12, (ii) a second gene expression level of a second human gene selected from the group consisting of genes shown in Table 12, wherein the second human gene is different from the first human gene, (c) detecting differences, if any, in the first gene expression level and the second gene expression level relative to reference expression levels characteristic of a human subject who is not infected with SARS-COV-2 and does not have signs or symptoms of COVID-19 disease, (d) determining whether the subject is infected with SARS-COV-2 based on the differences, if any, determined in step (c). In some approaches, the method further comprises detecting the presence or quantity of a SARS-COV-2 viral gene in the biological sample, and determining whether the subject is infected with SARS-COV-2 based on the detection the SARS-COV-2 viral gene and the differences, if any, determined in step (c).

Also provided herein is a method of diagnosing COVID-19 disease in a human subject, the method comprising (a) receiving a biological sample collected from the subject, the biological sample including human RNA from cells of the subject and SARS-COV-2 viral RNA, if present, (b) measuring, in the biological sample, (i) a first gene expression level of a first human gene selected from the group consisting of genes shown in Table 12, (ii) a second gene expression level of a second human gene selected from the group consisting of genes shown in Table 12, wherein the second human gene is different from the first human gene, (c) detecting differences in the first gene expression level and the second gene expression level relative to reference expression levels characteristic of a human subject who is not infected with SARS-COV-2 and does not have signs or symptoms of COVID-19 disease, (d) determining whether the subject has COVID-19 disease based on the differences, if any, determined in (c). In some approaches, the method further comprises detecting the presence or quantity of a SARS-COV-2 viral gene in the biological sample, and determining whether the subject has COVID-19 based on the detection of the SARS-COV-2 viral gene and the differences, if any, determined in step (c).

The SARS-COV-2 viral gene may be a SARS-COV-2 Envelope (E) gene, a SARS-COV-2 Nucleocapis (N) gene, a Spike (S) gene, an SARS-COV-2 open reading frame 1ab (Orf1ab) gene, or an SARS-COV-2 RNA dependent RNA polymerase (RdRP) gene. In some aspects, the SARS-COV-2 viral gene is an SARS-COV-2 N gene. In some aspects, the SARS-COV-2 viral gene is an SARS-COV-2 E gene.

In other aspects, the disclosure relates to a method of diagnosing COVID-19 in a subject, the method comprising (a) receiving a biological sample from the subject, the biological sample including RNA of the subject and potentially RNA of SARS-COV-2, (b) measuring, in the biological sample, a first gene expression level of a first human gene selected from the group consisting of genes shown in Table 12, (c) measuring, in the biological sample, a viral expression level of a SARS-COV-2 viral gene, (d) determining a classification of whether the subject has COVID-19 using the first gene expression level, the viral expression level, and one or more reference gene expression levels determined from a plurality of reference samples having a known COVID-19 classification. In another method, the expression level of any one of the genes as shown in Table 12 or Table 13 may be measured and be used to classify subjects without measuring expression level of a viral marker. Thus, further provided is a method of diagnosing COVID-19 in a subject, the method comprising (a) receiving a biological sample from the subject, the biological sample including RNA of the subject and potentially RNA of SARS-COV-2, (b) measuring, in the biological sample, a first gene expression level of a first human gene selected from the group consisting of genes shown in Table 12 or Table 13, (c) determining a classification of whether the subject has COVID-19 using the first gene expression level and one or more reference gene expression levels determined from a plurality of reference samples having a known COVID-19 classification.

Variations of the methods described herein further comprise measuring, in the biological sample, a second gene expression level of a second human gene selected from the group consisting of genes shown in Table 12 or Table 13, where the method comprises determining a classification of whether the subject has COVID-19 using the first gene expression level, the second gene expression level, the viral expression level, and one or more reference gene expression levels determined from a plurality of reference samples having a known COVID-19 classification. In some embodiments, where the expression level of a viral marker is not measured, the method comprises determining a classification of whether the subject has COVID-19 using the first gene expression level, the second gene expression level, and one or more reference gene expression levels determined from a plurality of reference samples having a known COVID-19 classification.

In other variations, the methods described herein further comprise measuring, in the biological sample, a third gene expression level of a second human gene selected from the group consisting of genes shown in Table 12 or Table 13, where the method comprises determining a classification of whether the subject has COVID-19 using the first gene expression level, the second gene expression level, the third expression level, the viral expression level, and one or more reference gene expression levels determined from a plurality of reference samples having a known COVID-19 classification. In some embodiments, where the expression level of a viral marker is not measured, the method comprises determining a classification of whether the subject has COVID-19 using the first gene expression level, the second gene expression level, the third expression level, and one or more reference gene expression levels determined from a plurality of reference samples having a known COVID-19 classification. In some embodiments, the methods provided herein include measuring the expression level of more than three genes selected from the group consisting of genes shown in Table 12 or Table 13. For example, in some embodiments, the methods described herein further comprise measuring, in the biological the gene expression level of a fourth, fifth, or sixth human gene selected from the group consisting of genes shown in Table 12 or Table 13.

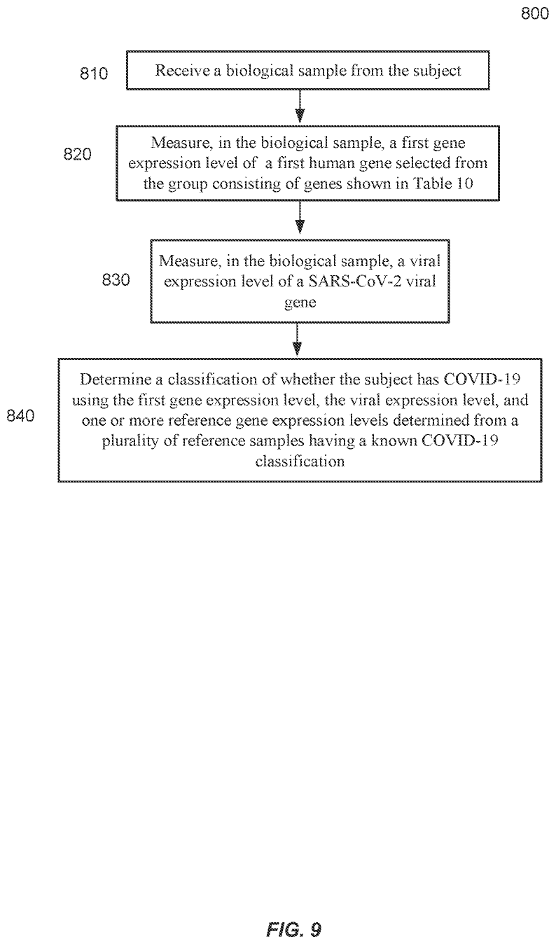

FIG. 9 is a flowchart illustrating a method of measuring the expression levels of the gene markers described herein to diagnose COVID-19 in a subject according to embodiments of the present invention.

In step 810, a biological sample is received from the subject. Any type of sample can be used from the individual. In some aspects, the biological sample is a sample comprising cells from the nose, mouth, or throat of the subject. In some aspects, the biological sample comprises cells collected from the subject's nose and/or mouth and/or throat. In some aspects, the sample is collected using a buccal swab, nasal swab, nasopharyngeal swab, nasopharyngeal wash or aspirate, mid-turbinate a sample from the nose or mouth. In some embodiments, the biological sample comprises fluid from the lungs, such as a broncho-alveolar lavage, or a tracheobronchial aspirate. In some embodiments, serum, plasma, or blood from the subject may be used as a sample. Tissue samples may also be used, such as a lung tissue. The biological sample includes nucleic acid molecules (e.g., RNA) of the subject and potentially viral RNA of SARS-COV-2. Nucleic acids (e.g., RNA) can be purified from the sample. General molecular biology methods that can be used are described, for example, in Sambrook and Russell, Molecular Cloning, A Laboratory Manual (3rd ed. 2001) and Ausubel F. M. et al. (Eds) Current Protocols in Molecular Biology (2007), John Wiley and Sons, Inc. Such nucleic acids may also be obtained through in vitro amplification methods such as those described herein and in PCR Protocols A Guide to Methods and Applications (Innis et al, eds) Academic Press Inc. San Diego, Calif. (1990) (Innis), incorporated by reference in its entirety for all purposes and in particular for all teachings related to amplification methods. In some embodiments, the nucleic acids will not be amplified before they are measured.

In step 820, a first gene expression level of a first human gene selected from the group consisting of genes shown in Table 12 or Table 13 is measured in the biological sample. In some embodiments, the method further comprises measuring, in the biological sample, a second gene expression level of a second human gene selected from the group consisting of genes shown in Table 12 or Table 13. In some embodiments, the method further comprises measuring, in the biological sample, a third gene expression level of a third human gene selected from the group consisting of genes shown in Table 12 or Table 13. Gene expression levels can be measured by any suitable method, such as those described in section VIII. In some preferred embodiments, amplification based methods (e.g., PCR or LAMP assays) or nucleotide sequencing techniques are used to measure and quantify gene expression levels.

In step 830, a viral expression level of a SARS-COV-2 viral gene is measured in the biological sample. Any suitable sequence of the SARS-COV-2 RNA may be used. Exemplary sequences include a sequence that encodes for the Nucleocapsid (N), Envelope (E), Spike (S) protein, the open reading frame 1ab (Orf1ab), and/or the RNA dependent RNA polymerase (RdRP) gene, which is located within Orf1ab.

In general, step 830 and step 840 can be performed simultaneously. In some embodiments, step 830 may be performed before step 820. For instance, measuring a viral expression level of a SARS-COV-2 viral gene can be performed prior to a first gene expression level of a first human gene.

Step 840 includes determining a classification of whether the subject has COVID-19 using the first gene expression level, the viral expression level, and one or more reference gene expression levels determined from a plurality of reference samples having a known COVID-19 classification. In some embodiments, determining a classification may further include using the second gene expression level. In some embodiments, determining a classification may further include using the third gene expression level. The classification can be binary or includes more levels, e.g., corresponding to a probability. Example classifications can include positive (i.e. SARS-COV-2 or COVID-19 detected), negative, and unclassified, as well as varying degrees of positive and negative (e.g., using integer numbers between 1 and 10, or real number between 0 and 1).

Reference samples may be samples of a SARS-COV-2 infected population and/or samples of a population without a SARS-COV-2 infection. In some embodiments, determining a classification can involve applying various statistical methods and/or machine learning techniques, such as supervised machine learning (e.g. decision trees, nearest neighbor, support vector machines, and neural networks) and unsupervised machine learning (e.g., clustering, principal component analysis, etc.). Reference samples can be used to determine reference gene expression levels, i.e., the expression level of a gene shown in Table 12 or Table 13 in the reference sample. Reference gene expression levels from the reference samples can then be used to determine a cutoff value that discriminates between different classifications. For example, determining the classification may include comparing the first gene expression level to a cutoff value that discriminates between different COVID-19 classifications, where the cutoff is determined using the one or more reference gene expression levels. In some embodiments, determining the classification includes inputting the first gene expression level to a machine learning model that discriminates between different COVID-19 classifications, and wherein the machine learning model is trained using the one or more reference gene expression levels and the known COVID-19 classifications of the plurality of reference samples. In some embodiments, where a second gene expression level is used, determining the classification may further include comparing the second gene expression level to a cutoff value that discriminates between different COVID-19 classifications, where the cutoff is determined using the one or more reference gene expression levels (reference gene expression levels for the second gene). In some embodiments, where a third gene expression level is used, determining the classification may further include comparing the third gene expression level to a cutoff value that discriminates between different COVID-19 classifications, where the cutoff is determined using the one or more reference gene expression levels (reference gene expression levels for the second gene). In some embodiments, where the viral expression level of a SARS-COV-2 viral gene is used, determining the classification may further include comparing the viral expression level to a cutoff value that discriminates between different COVID-19 classifications, where the cutoff is determined using the one or more reference gene expression levels of the viral marker.

The present disclosure also provides methods for determining a prognosis or assessing disease outcome in a subject having COVID-19. As such, prognosis refers to the likelihood of a clinical outcome for a subject afflicted with a SARS-COV-2 infection. Any of the gene markers listed in Table 12 or Table 13 may be used as a prognostic marker and indicator of disease progress and outcome. For example, the expression level of the genes provided herein may be predictive of disease severity. In some embodiments, the expression level of the genes provided herein may predict if the subject will have a mild illness (e.g., remain on outpatient treatment) or will develop a severe disease (e.g., require hospitalization). In some embodiments, the expression level of the genes provided herein may predict, for example, the development of respiratory failure requiring mechanical ventilation, the development of acute respiratory distress syndrome (ARDS), the duration of hospitalization, response to a treatment, and/or the mortality risk. In some embodiments, the host genes of the present disclosure may be useful for determining an increased risk of mortality within 30 days in a subject with COVID-19 or suspected of having COVID-19. Thus, the provided methods can be useful to determine if a subject should be monitored more closely for development of a severe illness so that appropriate treatment can be administered promptly and/or prophylactically.

The method of determining a prognosis in a subject having COVID-19 comprises the steps of (a) receiving a biological sample from the subject, the biological sample including RNA of the subject and potentially RNA of SARS-COV-2, (b) measuring, in the biological sample, a first gene expression level of a first human gene selected from the group consisting of genes shown in Table 12 or Table 13, (c) measuring, in the biological sample, a viral expression level of a SARS-COV-2 viral gene, and (d) determining a classification of disease outcome using the first gene expression level, the viral expression level, and one or more reference gene expression levels determined from a plurality of reference samples having a known disease outcome classification. The method of determining a prognosis in a subject having COVID-19 can be implemented in a similar manner as for the diagnostic method described above and illustrated in FIG. 9. Reference samples may be samples of a SARS-COV-2 infected population with a known disease phenotype outcome, such as disease severity, development of respiratory failure requiring mechanical ventilation, development of acute respiratory distress syndrome (ARDS), duration of hospitalization, response to a treatment, and/or mortality (e.g., 30 day mortality).

### VII. Machine Learning Techniques for Gene Selection

### A. Classifier Derivation and Validation Dataset

In order to select for patients early in the disease course, the combined dataset was filtered to remove samples from SARS-COV-2+ patients having a Cycle threshold (Ct) value >30. The Ct value indicates the number of cycles necessary in an amplification method (e.g., PCR) to detect the virus in a sample and can be used as an indication of how much virus the sample comprises. Generally, the lower the Ct the more virus is present in the sample. For the filtration we took advantage of the fact that there is an inverse linear relationship between log 2 reads per million (rpM) and Ct values. Specifically, based on data published in Mick et al. (2020), this relationship can be expressed by the following formula: log 2(rpM)=31.9753-0.9167*Ct

As a result, an rpM value of 22.23 is approximately the same as a Ct value of 30. Therefore, 46 SC+ samples with an rpM <22.23 were removed from the combined dataset, leaving 318 samples.

The filtered dataset was normalized using the variance stabilizing transformation (VST) from the DESeq2 package. Other normalization approaches were also explored, such as TSS, which normalizes data from each sample by dividing by the total counts across the entire sample, but VST produced the most consistent results and VST was therefore adopted as the standard approach. After normalization, the dataset was centered and scaled using the StandardScaler class in sklearn. This class applies a linear transformation to data for each feature using the following formula:

(x−u)/s

where x is the original data, u is mean of the feature data, s is the variance of the feature data.

The filtered, centered and scaled dataset (the “filtered combined dataset”) was randomly split into a training set (70% of samples) and a validation set (30% of samples), using a stratified split to ensure that each set contained a similar SARS-COV-2+:SARS-COV-2− ratio. The training set was used for identifying n-gene sets, and both cohorts, along with the Ramlall et al. dataset, were used for evaluating the performance of the n-gene sets.

### B. Selection of Host Gene Sets

### 1. Greedy Feature Selection Algorithm

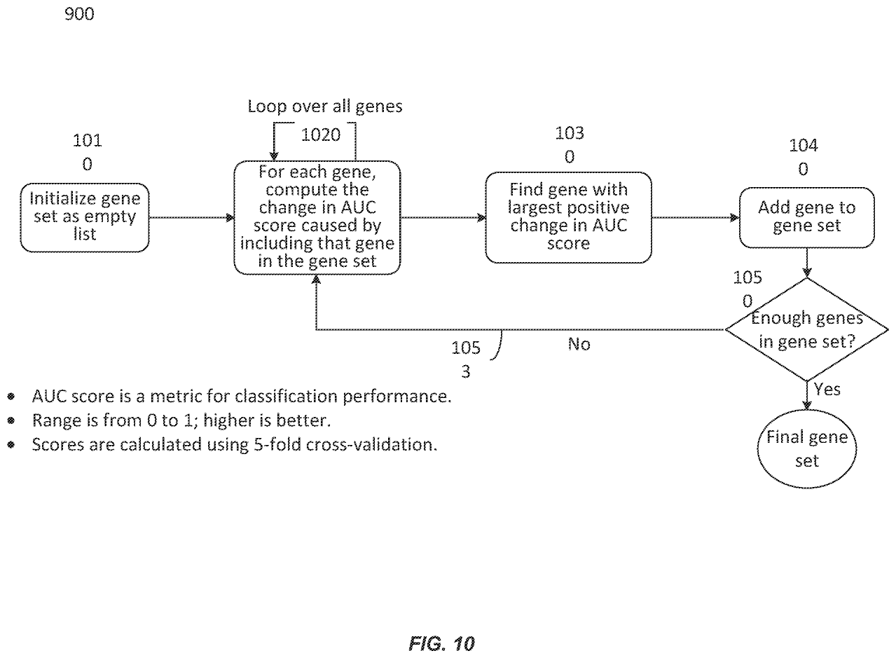

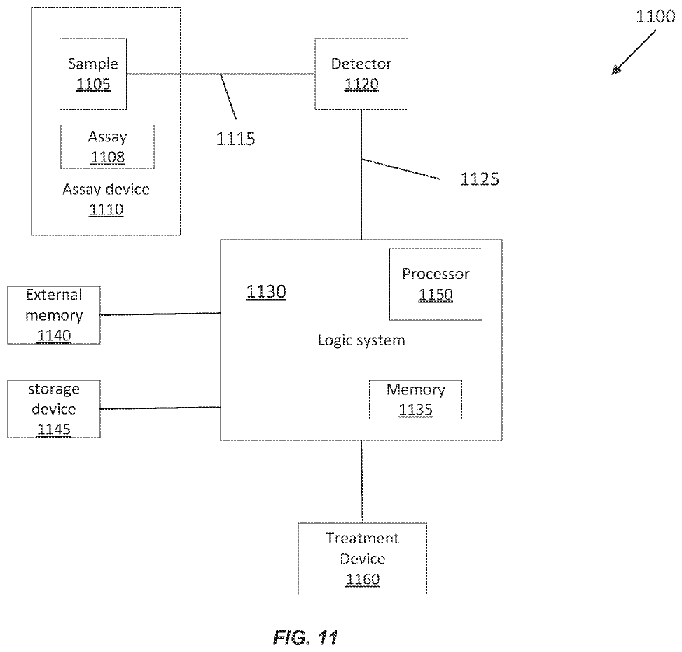

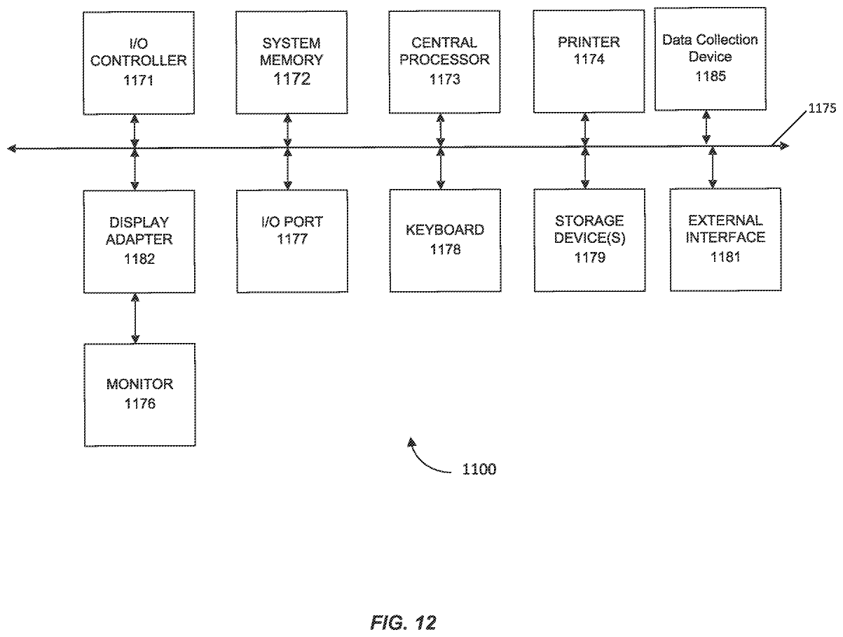

FIG. 10 illustrates an overall flow of a method 900 for gene set selection using a greedy feature selection algorithm. Gene sets were assembled one gene at time, using multiple rounds of the greedy feature selection algorithm to identify the next gene to add to the gene set.

At block 910 the gene set is initialized as an empty list. Thus, no assumptions are made as to what will be in the final gene set. In other implementations, the initial list can be seeded with one or more genes, which can be fixed or be removed in the process.

At block 920, during a first round, the algorithm iterates through every gene in the dataset, creating and evaluating a classifier, e.g., a support vector classifier (SVC), using the gene being tested as the sole feature. The output of block 920 is the AUC corresponding to each gene. At later stages, signified by line 953, the addition of each of the remaining genes is checked and an AUC is determined. For example, in a second round after selecting a first gene, the remaining N−1 genes are checked, and N−1 AUC values are output.

At block 930, the gene with the largest AUC is identified. In first round, this determination would be made for all N genes. In the second round, this determination would be made for the N−1 remaining genes.

At block 940, the gene with the largest AUC is added to the gene set. Blocks 930 and 940 show that in each subsequent round, the algorithm iterates through every gene in the dataset, creating and evaluating a classifier on a group of features consisting of the gene being tested plus the partial gene set identified in the previous round. In each case, the classifier is implemented in scikit-learn (www.scikit-learn.org) using the sklearn.svm.SVC class with default parameters, and performance of the SVC is evaluated by running 5-fold cross-validation on the 70% training set and calculating an AUC score.

In block 950, it is evaluated whether enough genes are in the gene set. This evaluation can be made in various ways, e.g., based on a predetermined number of genes to be added or based on a desired AUC. If the stopping criteria has not been satisfied, then method 900 can proceed to block 920.

In block 960 the final gene set is assembled.

To remove poorly or inconsistently performing gene sets, all gene sets with AUC scores above a threshold value are then re-evaluated using 10 rounds of 5-fold cross-validation on the 70% set. Any gene set that produces an AUC score below the threshold in any of the 10 rounds is eliminated from consideration. The threshold value is set by picking the AUC score corresponding to an ordinal position in the ranked list of gene sets. The ordinal position is determined empirically based on the total number of genes in the dataset and the distribution of AUC scores in the round. In the case of the combined dataset (i.e., Mick et al. dataset plus UCSF dataset), which has 15783 genes, the threshold was set at the 783rd highest AUC score for the first round of the algorithm, the 1783rd highest AUC score for the second round of the algorithm, and the 2783rd highest AUC score for all subsequent rounds. All gene sets that survive this thresholding process are then re-evaluated using 100 rounds of 5-fold cross-validation on the 70% set. The best-performing gene set at the end of this process (based on AUC score) is picked as the winner of the round. The above process is repeated multiple times until the desired n-gene set is identified.

In the case of two-gene sets, a slightly modified version of the algorithm was used in order to produce a diverse group of possible gene sets. At the end of the first round of the greedy selection algorithm, the top three best-performing single genes were identified. To extend these single-gene sets to two-gene sets, a second round of the algorithm was performed using each single gene set as the starting point, picking the top three best-performing 2-gene sets at the end of the round. As described above this resulted in a total of 9 two-gene sets (see Table 2 above).

In order to rigorously assess the performance of n-gene sets on the filtered combined dataset, SVC models using each n-gene set were evaluated in three ways: (1) running 10000 rounds of 5-fold cross-validation on the 70% training set and calculating the average AUC score and standard deviation, (2) running 10000 rounds of 5-fold cross-validation on the 30% validation set and calculating the average AUC score and standard deviation, and (3) training each model on the 70% training set and testing it on the 30% validation set to generate an AUC score. The same evaluations were performed using the unfiltered version of the combined dataset.

### 2. Intersection Approach

As an alternative approach to identifying genes that might be useful in distinguishing SARS-COV-2+ and SARS-COV-2− samples by PCR, the greedy selection algorithm was combined with Differential Expression (DE) analysis as follows. First, a single round of the gene selection algorithm was used to generate a ranked list of all genes based on each gene's performance on the filtered combined dataset. As described in section VII.B.1, the algorithm iterates through every gene in the dataset, creating and evaluating a support vector classifier (SVC) using the gene being tested as the sole feature. The SVC is implemented in scikit-learn (www.scikit-learn.org) using the sklearn.svm.SVC class with default parameters, and performance of the SVC is evaluated by running 5-fold cross-validation on the 70% training set and calculating an AUC score. However, for this alternative approach, rather than using the thresholding process described in section VII.B.1, an average AUC score was obtained by performing five rounds of 5-fold cross-validation for each gene. The same process was also applied to the Ramlall et al. dataset in parallel, thus producing two independent lists (see Table 7 above). A second round of the gene selection algorithm was then performed using the top 5 best performing genes as the starting point in order to generate ranked lists of second genes.

DE analysis was performed separately on the Mick et al. dataset and the Ramlall et al. dataset. Results of these analyses were compared with the list of first and second genes from the greedy selection algorithm. The list of first genes was highly correlated with genes that are differentially expressed in the SARS-COV-2+ samples relative to the “no virus” samples, while the list of second genes was highly correlated with genes that are differentially expressed in the SARS-COV-2+ samples relative to the “other virus” samples. This observation is consistent with the gene expression patterns described above in section IV.A (see description of FIGS. 3A-3D and 4A-4D). A list of candidate genes for PCR was selected based on the comparison of the DE analyses with the list of first and second genes.

### VIII. Measuring Gene Expression Levels

Techniques and methods for measuring the expression levels of human genes and for detecting viral RNA (e.g., SARS-COV-2 RNA) are available in the art. For example, measuring the expression level of genes listed in Table 12 or Table 13 and the detection of viral RNA may be accomplished by any suitable amplification method, such as polymerase chain reaction (PCR) methods and isothermal amplification methods (see section VIII.A and VIII.B below). Isothermal amplification methods that may be used to measure gene expression levels include, for example, loop-mediated isothermal amplification (LAMP). In some approaches, sequencing technologies may be used to quantify gene expression levels (e.g., metagenomic next generation sequencing; described in section VIII.C, below). Other methods that may be used for measuring gene expression levels include but are not limited to hybridization capture methods, microarray analysis, Northern blot, serial analysis of gene expression (SAGE), and immunoassays. These methods are described, for example, in Sambrook and Russel (2001), Molecular Cloning: A Laboratory Manual, 3rd Edition, Cold Spring Harbor, N.Y.: Cold Spring Harbor Laboratory Press; Velculescu et al., 1995, Science 270:484-7; Serial Analysis of Gene Expression (SAGE): Methods and Protocols (Methods in Molecular Biology), Humana Press, 2008; herein incorporated by reference in their entireties.

### A. Methods Based on Polymerase Chain Reaction (PCR)

In some approaches, a polymerase chain reaction (PCR) may be used to measure the gene expression levels. In some approaches, polymerase chain reaction (PCR) may be used to detect SARS-COV-2 RNA. PCR-based methods that may be used include but are not limited to quantitative PCR (qPCR or real-time PCR), reverse transcriptase PCR (RT-PCR), and digital PCR. PCR methods are well known in the art, and are described, for example, in Innis et al., eds., PCR Protocols: A Guide To Methods And Applications, Academic Press Inc., San Diego, Calif. (1990); see Sambrook and Russel (2001), Molecular Cloning: A Laboratory Manual, 3rd Edition, Cold Spring Harbor, N.Y.: Cold Spring Harbor Laboratory Press; Chapter 8: In vitro Amplification of DNA by the Polymerase Chain Reaction; PCR Technology: Principles and Applications for DNA Amplification (ed. H. A. Erlich, Freeman Press, N.Y., N.Y., 1992, herein incorporated by reference in their entirety.

In some approaches, quantitative reverse transcriptase PCR (qRT-PCR) may be used. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMY-RT) and Moloney murine leukemia virus reverse transcriptase (MLVRT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction. Although the PCR step can use a variety of thermostable DNAdependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, may be designed to detect nucleotide sequence located between the two PCR primers. The probe is non-extendible by Taq DNA polymerase enzyme, and may be labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. See, e.g. Real-Time PCR: Current Technology and Applications, Logan, Edwards, and Saunders eds., Caister Academic Press, 2009; Joyce (2002), “Quantitative RT-PCR. A review of current methodologies,” Methods Mol. Biol. 193. pp. 83-92; Bustin et al. (2005), “Quantitative real-time RT-PCR—a perspective,” J. Mol. Endocrinol. 34 (3): 597-601; Bustin (2000), “Absolute quantification of mRNA using real-time reverse transcription polymerase chain reaction assays,” J. Mol. Endocrinol. 25 (2): 169-93; Deepak et al. (2007), “Real-Time PCR: Revolutionizing Detection and Expression Analysis of Genes”. Curr. Genomics. 8 (4): 234-51; Gause et al. (1994). “The use of the PCR to quantitate gene expression”. PCR Methods Appl. 3 (6): S123-35.

Accordingly, in some approaches measuring the expression level of the one or more genes shown in Table 12 or Table 13 comprises performing PCR (e.g., qRT-PCR). The PCR may be performed by using at least one set of oligonucleotide primers comprising a forward primer and a reverse primer capable of amplifying a polynucleotide sequence of the gene (such as IFI6). Methods for the design and/or production of nucleotide primers are generally known in the art, and are described in e.g., Sambrook et al. (2001) Molecular Cloning: A Laboratory Manual (3rd ed., Cold Spring Harbor Laboratory Press, Plainview, N.Y.); Ausubel F. M. et al. (Eds) Current Protocols in Molecular Biology (2007), John Wiley and Sons, Inc; Molecular Cloning: A Laboratory Manual, 4th ed., Green and Sambrook, 2012). Nucleotide primers and probes may be prepared, for example, by chemical synthesis techniques for example, the phosphodiester and phosphotriester methods (see for example Narang S. A. et al. (1979) Meth. Enzymol. 68:90; Brown, E. L. (1979) et al. Meth. Enzymol. 68:109; and U.S. Pat. No. 4,356,270), the diethylphosphoramidite method (see Beaucage S. L et al. (1981) Tetrahedron Letters, 22:1859-1862). Oligonucleotide primers are typically being between 5-80 nucleotides in length, e.g., between 10-50 nucleotides in length, or between 15-30 nucleotides in length. Any appropriate length of sequence may be used such as 10, 11, 12, 13, 14, 15, 16, 17, 18, 19, 20, 21, 22, 23, 24, 25, 26, 27, 28, 29, or 30 nucleotides or more.

For the detection of SARS-COV-2 RNA by RT-qPCR a number of primer and probe sets are known and are available e.g., at the Centers for Disease Control and Prevention (CDC; US Centers for Disease Control and Prevention. 2019-novel coronavirus (2019-nCoV) real-time rRTPCR panel primers and probes. Washington (DC): Department of Health and Human Services; www.who.int/docs/default-source/coronaviruse/uscdcrt-pcr-panel-primer-probes.pdf?sfvrsn=fa29cb4b_2). In addition, PCR-based methods for the detection of SARS-COV-2 RNA are described e.g., in Corman et al. (2020), “Detection of 2019 novel corovirus (2019-nCoV) by real-time RT-PCR, Euro. Surveill. 25, 2000045; and Kilic et al. (2020), “Molecular and immunological diagnostic tests of COVID-19: Current status and challenges, iScience, 23(8): 101406; and Kudo et al. (2020), “Detection of SARS-COV-2 RNA by multiplex RT-qPCR, PLoS Biol. 2020 October; 18(10): e3000867.

### B. Isothermal Amplification Methods

In some embodiments, isothermal amplification methods can be used to measure the expression level of the genes. A number of isothermal amplification methods are known in the art and have been discussed, e.g., in Zhao et al. (2015), “Isothermal amplification of nucleic acids,” Chem. Rev., 115 (22), 12491-12545; Niemz et al. (2011), “Point-of-care nucleic acid testing for infectious diseases,” Trends Biotechnol.; 29:240-250; Yan et al. 92014), “Isothermal amplified detection of DNA and RNA,” Mol. Biosyst. 10, 970-1003. Any suitable isothermal amplification method may be used. In some approaches, loop-mediated isothermal amplification (LAMP) may be used. For example, LAMP may be particularly suitable for point of care (POC) settings as the method typically operates at 60-65° C. to achieve exponential amplification of nucleic acid targets without requiring temperature cycling. LAMP methods are known in the art and described, e.g., in U.S. Pat. No. 6,410,278; Notomi et al. (2000), “Loop-mediated isothermal amplification of DNA,” Nucleic Acids Res.; 28:E63; Nagamine et al. (2002), “Accelerated reaction by loop-mediated isothermal amplification using loop primers,” Mol. Cell. Probes. 16 (3): 223-9; Tomita et al. (2008), “Loop-mediated isothermal amplification (LAMP) of gene sequences and simple visual detection of products,” Nat. Protoc. 3, 877-82; Fu et al. (2011), “Applications of loop-mediated isothermal DNA amplification,” Appl. Biochem. Biotechnol. 163, 845-50. LAMP is a one-step amplification system using auto-cycling strand displacement DNA synthesis. The target sequence is amplified at a constant temperature of 60-65° C. using either two or three sets of primers and a polymerase with high strand displacement activity in addition to a replication activity. Typically, 4 different primers are used to amplify 6 distinct regions on the target gene, which increases specificity. An additional pair of “loop primers” can further accelerate the reaction. The amplification product can be detected via photometry, measuring the turbidity caused by magnesium pyrophosphate precipitate in solution as a byproduct of amplification.

Other isothermal amplification methods that may be used include but are not limited to transcription-mediated amplification (TMA) Nucleic Acid Sequence Based Amplification (NASBA), Multiple Displacement Amplification (MDA), Rolling Circle Amplification (RCA), Helicase Dependent Amplification (HDA), Strand Displacement Amplification (SDA), Nicking Enzyme Amplification Reaction (NEAR), Ramification Amplification Method (RAM), and Recombinase Polymerase Amplification (RPA). In some approaches, TMA is used to measure the expression level of the genes (and potentially SARS-COV-2 RNA).

Isothermal amplification methods for measuring host gene expression levels may be used in conjunction with isothermal amplification methods (e.g., LAMP assays) detecting SARS-COV-2 RNA. For example, several LAMP assays have been developed to target different gene regions of SARS-COV-2,with fluorescence or colorimetric readouts. See e.g., Yan et al. (2020), “Rapid and visual detection of 2019 novel coronavirus (SARS-COV-2) by a reverse transcription loop-mediated isothermal amplification assay,” Clin. Microbiol. Infect. 26, 773-779; Zhang et al. (2020), “Rapid molecular detection of SARS-COV-2 (COVID-19) virus RNA using colorimetric LAMP,” medRxiv; Zhu et al. (2020), “Reverse transcription loop-mediated isothermal amplification combined with nanoparticles based biosensor for diagnosis of COVID-19,” medRxiv; Yu et al. (2020). Rapid colorimetric detection of COVID-19 coronavirus using a reverse transcriptional loop-mediated isothermal amplification (RT-LAMP) diagnostic platform: iLACO,” medRxiv; Lu et al. (2020), “Development of a novel reverse transcription loop-mediated isothermal amplification method for rapid detection of SARS-COV-2. Virol. Sin. 1-4.

### C. Sequencing Technologies

The gene expression levels may be measured using sequencing technologies, such as next generation sequencing platforms (e.g., RNA-Seq). RNA-SEQ uses next-generation sequencing (NGS) for the detection and quantification of RNA in a biological sample at a given moment in time. An RNA library is prepared, transcribed, fragmented, sequenced, reassembled and the sequence or sequences of interest quantified. NGS methods are well known in the art and described e.g., in Mortazavi et al., Nat. Methods 5: 621-628, 2008; Karl et al. (2009), “Next-Generation Sequencing: From Basic Research to Diagnostics,” Clinical Chemistry. 55 (4): 641-658; Wang et al. (2009), “RNA-Seq: a revolutionary tool for transcriptomics,” Nature Reviews. Genetics. 10 (1): 57-63; Kukurba and Montgomery (2015), “RNA Sequencing and Analysis”, Cold Spring Harbor Protocols., (11): 951-69. In some approaches, whole transcriptome shotgun sequencing may be used to measure gene expression levels. In some approaches, metagenomics NGS (mNGS) may be used to measure gene expression levels. See e.g., Chiu and Miller (2019), “Clinical metagenomics,” Nature Reviews Genetics, 20 (6): 341-355; Maljkovic et al. (2019), “Next Generation Sequencing and Bioinformatics Methodologies for Infectious Disease Research and Public Health: Approaches, Applications, and Considerations for Development of Laboratory Capacity,” The Journal of Infectious Diseases: jiz286; Wilson et al. (2019), “Clinical metagenomic sequencing for diagnosis of meningitis and encephalitis,” N. Engl. J. Med. 380, 2327-2340. Exemplary sequencing platforms suitable for use according to the methods include, e.g., ILLUMINA® sequencing (e.g., HiSeq, MiSeq), SOLID® sequencing, ION TORRENT® sequencing, and SMRT® sequencing and those commercialized by Roche 454 Life Sciences (GS systems).

In some embodiments, mNGS may be used to determine host gene expression levels in conjunction with the quantification of SARS-COV-2 RNA abundance. For example, quantification of SARS-COV-2 RNA may involve aligning to the SARS-COV-2 reference genome (e.g., MN908947.3) using minimap2 (described in Li at al. (2018), “Minimap2: pairwise alignment for nucleotide sequences,” bioinformatics, 34, 3094-3100) and calculating SARS-COV-2 reads per million (rpM) using the number of reads that aligned with mapq >=20. See e.g., Mick et al. (2020).

### IX. Computer Systems

Embodiments can provide a method for determining a classification of the presence or absence for COVID-19 and/or determine a prognosis for a subject having COVID-19. In some instances, the method can be performed by a computer system and/or a measurement system. In some embodiments, expression level data can be received at the computer system, e.g., from a detection or measuring apparatus, such as a PCR device or a sequence machine that provides data to a storage device (which can be loaded into the computer system) or across a network to the computer system. The received data can then be analyzed, interpreted and visualized by the computer system. In some examples, the present disclosure provides systems, methods, or kits that can include data analysis realized in measurement devices (e.g., laboratory instruments, such as a PCR device or sequencing machine).

FIG. 11 illustrates a measurement system 1000 according to an embodiment of the present disclosure. The system as shown includes a sample 1005, such as a biological sample comprising RNA molecules within an assay device 1010, where an assay 1008 can be performed on sample 1005. For example, sample 1005 can be contacted with reagents of assay 1008 to provide a signal of a physical characteristic 1015. An example of an assay device can be a PCR devise that includes probes and/or primers. Physical characteristic 1015 (e.g., a fluorescence intensity, a voltage, or a current), from the sample is detected by detector 1020. Detector 1020 can take a measurement at intervals (e.g., periodic intervals) to obtain data points that make up a data signal. In one embodiment, an analog-to-digital converter converts an analog signal from the detector into digital form at a plurality of times. Assay device 1010 and detector 1020 can form an assay system, e.g., a sequencing system that performs sequencing according to embodiments described herein. A data signal 1025 is sent from detector 1020 to logic system 1030. As an example, data signal 1025 can be used to determine sequences and/or locations in a reference genome of RNA molecules. Data signal 1025 can include various measurements made at a same time, e.g., different colors of fluorescent dyes or different electrical signals for different molecule of sample 1005, and thus data signal 1025 can correspond to multiple signals. Data signal 1025 may be stored in a local memory 1035, an external memory 1040, or a storage device 1045.

Logic system 1030 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1030 and the other components may be part of a stand-alone or network connected computer system, or they may be directly attached to or incorporated in a device (e.g., a sequencing device) that includes detector 1020 and/or assay device 1010. Logic system 1030 may also include software that executes in a processor 1050. Logic system 1030 may include a computer readable medium storing instructions for controlling measurement system 1000 to perform any of the methods described herein. For example, logic system 3930 can provide commands to a system that includes assay device 1010 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

System 1000 may also include a treatment device 1060, which can provide a treatment to the subject. Treatment device 1060 can determine a treatment and/or be used to perform a treatment. Examples of such treatment can include surgery, anti-viral therapies, anti-inflammatory therapies, immunotherapy, hormone therapy, and stem cell transplant. Logic system 1030 may be connected to treatment device 1060, e.g., to provide results of a method described herein. The treatment device may receive inputs from other devices, such as an imaging device and user inputs (e.g., to control the treatment, such as controls over a robotic system).

Any of the computer systems mentioned herein may utilize any suitable number of subsystems. Examples of such subsystems are shown in FIG. 12 in computer system 1100. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 12 are interconnected via a system bus 1175. Additional subsystems such as a printer 1174, keyboard 1178, storage device(s) 1179, monitor 1176 (e.g., a display screen, such as an LED), which is coupled to display adapter 1182, and others are shown. Peripherals and input/output (I/O) devices, which couple to I/O controller 1171, can be connected to the computer system by any number of means known in the art such as input/output (I/O) port 1177 (e.g., USB, FireWire®). For example, I/O port 1177 or external interface 1181 (e.g. Ethernet, Wi-Fi, etc.) can be used to connect logic system 1030 to a wide area network such as the Internet, a mouse input device, or a scanner. The interconnection via system bus 1175 allows the central processor 1173 to communicate with each subsystem and to control the execution of a plurality of instructions from system memory 1172 or the storage device(s) 1179 (e.g., a fixed disk, such as a hard drive, or optical disk), as well as the exchange of information between subsystems. The system memory 1172 and/or the storage device(s) 1179 may embody a computer readable medium. Another subsystem is a data collection device 1185, such as a camera, microphone, accelerometer, and the like. Any of the data mentioned herein can be output from one component to another component and can be output to the user.

A computer system can include a plurality of the same components or subsystems, e.g., connected together by external interface 1181, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Steps of methods described herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

### X. Kits and Devices

In another aspect, provided in this disclosure are kits, panels and devices for carrying out the methods described herein. In some embodiments, a kit is provided for measuring and analyzing RNA in a biological sample. The kit can comprise one or more polynucleotides for specifically hybridizing to at least a section of a gene listed in Table 12 or Table 13. In one embodiment, the kit includes two or more polynucleotides for specifically hybridizing to at least a section of a gene listed in Table 12 or Table 13 for use in testing a subject for COVID-19. In another embodiment, the kit includes two or more polynucleotides for use in determining a prognosis in a subject having COVID-19, e.g., to determine a disease severity. In one aspect, provided herein is a medical or diagnostic device that can, for example, measure gene expression levels and provide a color indication when the gene marker(s) of interest shows differential gene expression levels in a subject. The device could be used in a clinical setting to determine if a subject has COVID-19.

In some embodiments, a kit or a panel as provided herein includes a reference sample, such as a sample from a healthy subject not infected with SARS-COV-2. In some embodiments, a kit or a panel as provided herein includes a reference sample, such as a sample from an infected subject having COVID-19. If such a sample is included, the measurement values (reference gene expression levels) for such sample are compared with the results of the test sample, so that the presence or absence of COVID-19 condition in the subject can be determined.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects. The above description of example embodiments of the invention has been presented for the purposes of illustration and description. It is not intended to be exhaustive or to limit the invention to the precise form described, and many modifications and variations are possible in light of the teaching above.

All patents, patent applications, publications, and descriptions mentioned herein are incorporated by reference in their entirety for all purposes. None is admitted to be prior art.

1. A method of determining whether or not a human subject is infected with SARS-COV-2, the method comprising:
(a) receiving a biological sample collected from the human subject, the biological sample including human RNA from cells of the human subject;
(b) measuring, in the biological sample,
i) a first gene expression level of a first human gene selected from the group consisting of genes shown in Table 12A or Table 13A;
ii) a second gene expression level of a second human gene selected from the group consisting of genes shown in Table 12B or Table 13B, wherein the second human gene is different from the first human gene;
(c) detecting differences, if any, in the first gene expression level and the second gene expression level relative to reference expression levels characteristic of another human subject who is not infected with SARS-COV-2; and
(d) determining whether the human subject is infected with SARS-COV-2 based on the differences, if any, determined in (c).

2. The method of claim 1 wherein the human subject does not have signs or symptoms of COVID-19 disease.

3. The method of claim 1 wherein the human subject has signs or symptoms of COVID-19 disease.

4. The method of claim 1, wherein the biological sample comprises SARS-COV-2 viral RNA, and wherein a presence of the SARS-COV-2 viral RNA is detected.

5-6. (canceled)

7. The method of claim 4, further comprising detecting a presence or quantity of a SARS-COV-2 viral gene in the biological sample, and determining whether the human subject is infected with SARS-COV-2 based on the detection of the SARS-COV-2 viral gene and the differences, if any, determined in step (c).

8. The method of claim 2, further comprising:
detecting a presence or quantity of a SARS-COV-2 viral gene in the biological sample, and determining whether the human subject has COVID-19 based on the detection of the SARS-COV-2 viral gene and the differences, if any, determined in step (c).

9. The method of claim 1, wherein the first gene expression level and the second gene expression level in the biological sample are determined by normalizing a measured expression level with an expression level of a human normalization gene.

10. The method of claim 1, wherein the biological sample comprises cells collected from one or more of the subject's nose, mouth, throat, and lower respiratory tract.

11. The method of claim 7, wherein the SARS-COV-2 viral gene is a SARS-COV-2 Envelope (E) gene, a SARS-COV-2 Nucleocapsid (N) gene, a SARS-COV-2 Spike (S) gene, a SARS-COV-2 open reading frame 1ab (Orf1ab) gene, or a SARS-COV-2 RNA dependent RNA polymerase (RdRP) gene.

12. The method of claim 1, wherein the first human gene is IFI6, IFI44, IFI44L,_gr IFI27 and the second human gene is ANKRD22, CCL3, CD274, GBP5, GSTA2, PTAFR, SIGLEC10, or TIMP1.

13. A method of diagnosing COVID-19 disease in a human subject, the method comprising:
(a) measuring, in a biological sample from the human subject, which biological sample includes RNA of the human subject and potentially RNA of SARS-COV-2, a first gene expression level of a first human gene selected from the group consisting of genes shown in Table 13A;
(b) measuring, in the biological sample, a viral expression level of a SARS-COV-2 viral gene; and
(c) determining a classification of whether the human subject has COVID-19 using the first gene expression level, the viral expression level, and one or more reference gene expression levels determined from a plurality of reference samples having a known COVID-19 classification.

14. The method of claim 13, wherein step (a) further comprises measuring, in the biological sample, a second gene expression level of a second human gene selected from the group consisting of genes shown in Table 12B or Table 13B; and wherein (c) comprises determining the classification of whether the human subject has COVID-19 using the first gene expression level, the second gene expression level, the viral expression level, and the one or more reference gene expression levels determined from the plurality of reference samples having the known COVID-19 classification.

15. A method of diagnosing COVID-19 in a human subject, the method comprising:
(a) receiving a biological sample obtained from the human subject, the biological sample including RNA of the human subject;
(b) measuring, in the biological sample, a first gene expression level of a first human gene selected from the group consisting of genes shown in Table 13A; and
(c) determining a classification of whether the human subject has COVID-19 using the first gene expression level and one or more reference gene expression levels determined from a plurality of reference samples having a known COVID-19 classification.

16. The method of claim 15, wherein step (b) further comprises measuring, in the biological sample, a second gene expression level of a second human gene selected from the group consisting of genes shown in Table 12B or Table 13B; and wherein step (c) comprises determining the classification of whether the human subject has COVID-19 using the first gene expression level, the second gene expression level, and the one or more reference gene expression levels determined from the plurality of reference samples having the known COVID-19 classification.

17-20. (canceled)

21. The method of claim 1, wherein the biological sample is obtained using a nasal swab, a nasopharyngeal swab, an oropharyngeal swab, a buccal swab, a broncho-alveolar lavage, or an endotracheal aspirate.

22. The method of claim 14, wherein measuring the first gene expression level and the second gene expression level and measuring the viral expression level comprises performing polymerase chain reaction (PCR), isothermal amplification, next generation sequencing (NGS), microarray analysis, Northern blot, or serial analysis of gene expression (SAGE).

23. The method of claim 13, wherein determining the classification includes comparing the first gene expression level to a cutoff value that discriminates between different COVID-19 classifications, and wherein the cutoff value is determined using the one or more reference gene expression levels.

24. The method of claim 23, wherein determining the classification includes inputting the first gene expression level to a machine learning model that discriminates between different COVID-19 classifications, and wherein the machine learning model is trained using the one or more reference gene expression levels and the known COVID-19 classification of the plurality of reference samples.

25. The method of claim 1, wherein the first human gene and the second human gene are a two-gene set selected from two-gene sets in Table 12C.

26. The method of claim 1, wherein the first human gene and the second human gene are a two-gene set selected from two-gene sets in Table 13C.
